(19) United States
(12) Patent Application Publication
(10) Pub. No.: US 2018/0193153 A1
(43) Pub. Date: Jul. 12, 2018

(54) ARTICULAR SURFACE REPAIR

(71) Applicant: CONVENTUS ORTHOPAEDICS, INC., Maple Grove, MN (US)

(72) Inventors: Michael P. Brenzel, St. Paul, MN (US); Steve D. Kruse, St. Michael, MN (US); Todd A. Krinke, Buffalo, MN (US); Paul Hindrichs, Plymouth, MN (US); Jonathan Berndt, Crystal, MN (US)

(21) Appl. No.: 15/867,532

(22) Filed: Jan. 10, 2018

Related U.S. Application Data
(60) Provisional application No. 62/444,699, filed on Jan. 10, 2017.

(51) Int. Cl. A61F 2/30; A61F 2/46

(52) U.S. Cl. CPC A61F 2/30771; A61F 2/4603; A61F 2002/30667; A61F 2230/0069; A61F 2002/30624; A61F 2/30756

(57) ABSTRACT

Apparatus and methods for repairing a bone. The apparatus may include a prosthesis. The prosthesis may have a first face. The first face may be shaped to conform to a native articular bone surface and define a perimeter. The prosthesis may have a second face. The second face may be shaped to conform to a bone surface prepared for receiving the prosthesis. The second face may define two or more screw-holes adjacent the perimeter. The prosthesis may define, other than the screw-holes, no anchor pass-through.

### CROSS REFERENCE TO RELATED APPLICATIONS

This application is a nonprovisional of U.S. Provisional Application No. 62/444,699, filed on Jan. 10, 2017, which is hereby incorporated herein by reference in its entirety.

### BACKGROUND

Replacing an articular surface of a bone typically includes removing articular surface to form a shaped surface and placing a prosthesis on the shaped surface. A prosthesis support may be used in conjunction with the prosthesis. Conventional prosthesis supports include suture holes but not screw holes.

The bone receiving the prosthesis may be fractured. Conventional prosthesis supports typically cannot be used by a practitioner to reduce the fracture using screws.

It would be desirable, therefore, to provide apparatus and methods that enable a practitioner to provide therapy for an articular surface.

It would be desirable also, therefore, to provide apparatus and methods that enable a practitioner to provide therapy for an articular surface that is associated with fractured bone.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows illustrative apparatus and methods in accordance with principles of the invention.

FIG. 2 shows schematically illustrated apparatus in accordance with principles of the invention.

FIG. 3 shows schematically illustrated apparatus in accordance with principles of the invention.

FIG. 4 shows schematically illustrated apparatus in accordance with principles of the invention.

FIG. 5 shows schematically illustrated apparatus in accordance with principles of the invention.

FIG. 6 shows schematically illustrated apparatus in accordance with principles of the invention.

FIG. 7 shows schematically illustrated apparatus in accordance with principles of the invention.

FIG. 8 shows illustrative apparatus and methods in accordance with principles of the invention.

FIG. 9 shows conceptually a relationship between apparatus, in accordance with the principles of the invention, and anatomy.

FIG. 10 shows a human skeleton.

FIGS. 11 and 12 shows portions, in part, of a humerus bone.

FIG. 12 shows illustrative apparatus and methods in accordance with principles of the invention.

FIG. 13 shows illustrative apparatus and methods in accordance with principles of the invention.

FIG. 14 shows illustrative apparatus and methods in accordance with principles of the invention.

FIG. 15 shows illustrative apparatus and methods in accordance with principles of the invention.

FIG. 16 shows illustrative apparatus and methods in accordance with principles of the invention.

FIG. 17 shows illustrative apparatus and methods in accordance with principles of the invention.

FIG. 18 shows illustrative apparatus and methods in accordance with principles of the invention.

FIG. 19 shows illustrative apparatus and methods in accordance with principles of the invention.

FIG. 20 shows illustrative apparatus and methods in accordance with principles of the invention.

FIG. 21 shows illustrative apparatus and methods in accordance with principles of the invention.

FIG. 22 shows illustrative apparatus and methods in accordance with principles of the invention.

FIG. 23 shows illustrative apparatus and methods in accordance with principles of the invention.

FIG. 24 shows illustrative apparatus and methods in accordance with principles of the invention.

FIG. 25 shows illustrative apparatus and methods in accordance with principles of the invention.

FIG. 26 shows illustrative apparatus and methods in accordance with principles of the invention.

FIG. 27 shows illustrative apparatus and methods in accordance with principles of the invention.

FIG. 28 shows a cross-sectional view of apparatus illustrated in FIG. 27.

FIG. 29 shows illustrative apparatus and methods in accordance with principles of the invention.

FIG. 30 shows a cross-sectional view of apparatus illustrated in FIG. 29

FIG. 31 shows illustrative apparatus and methods in accordance with principles of the invention.

FIG. 32 shows an illustrative prosthesis in accordance with principles of the invention.

FIG. 33 shows an illustrative prosthesis in accordance with principles of the invention.

FIG. 34 shows an illustrative prosthesis in accordance with principles of the invention.

### DETAILED DESCRIPTION

Apparatus and methods for articular surface therapy are provided.

Apparatus and methods for fracture reduction are provided.

The apparatus may include a prosthesis. The apparatus may include an anchoring hull.

For the purposes of the application, “prosthesis” refers to an article that does not include monolithically or unitarily a screw. For the purposes of the application, “anchoring hull” refers to an article that does not include monolithically or unitarily a screw.

The prosthesis may be spaced apart, in its entirety, from the anchoring hull when the prosthesis and the anchoring hull are placed in therapeutic positions. Other than screws, there may be no therapeutic fixation that bridges from the prosthesis to the cage.

The prosthesis may abut the anchoring hull when the prosthesis and the anchoring hull are placed in therapeutic positions. The abutment may be an abutment that does not provide engagement between the prosthesis and the hull.

The prosthesis may be formed from ultra-high density polyethylene, metal, ceramic, pyrolytic carbon, nitinol, polymer, or any other suitable material used for prosthetic joints. The material may be extruded, machined, sintered, molded, or prepared by any other suitable process.

The prosthesis may include a replacement articular surface. The replacement articular surface may correspond to part or all of a native articular surface. The replacement articular surface may include all or some of the topography that is typically present in the native articular surface. The replacement articular surface may be referred to alternately herein as a “first face” of the prosthesis.

The prosthesis may include a seating surface that conforms to a prepared bone surface on the bone. The seating surface may seat on the prepared bone surface. The seating surface may be referred to alternately herein as a “second face” of the prosthesis.

A practitioner may shape the prepared bone surface to distribute force to be received from the prosthesis when the prosthesis is in use in the body. Native articular bone may be removed to shape the prepared bone surface using a reaming instrument, rongeurs, a saw, an osteotome, or any other apparatus used by those skilled in the art to remove native articular bone.

The practitioner may shape the prepared bone surface to counteract forces that urge the prosthesis to rotate relative to the prepared bone surface. For example, the prepared bone surface may be prepared to define a lineament (such as a ridge or valley). The seating surface may contour the prepared bone surface so that the lineament counteracts the rotation.

The prosthesis may be implemented in any suitable anatomical joint (in the human or animal body).

Exemplary human joints include the neck joint, intervertebral joint, shoulder joint, elbow joint, wrist joint, finger joint, sacroiliac joint, hip joint, knee joint and ankle joint. The joint may be a synovial joint. The joint may be a unilateral, biaxial or multiaxial joint. Table 1 shows illustrative types of the synovial joint.

TABLE 1
Illustrative types of the synovial joint.
Illustrative type of joint

Pivot
Hinge
Condyloid
Saddle
Plane
Ball and Socket

The native articular surface may include one or more of a convex region, a concave region, an oval region, a round region, and a flat region.

The seating surface may include one or more of a convex region, a concave region, an oval region, a round region, a flat region, a cone (in positive or negative relief), a region shaped like half of a torus (sliced along a diameter), a cylindrical region (longitudinal axis oriented perpendicular to the prepared surface or longitudinal axis lying in or on the prepared surface (and only part of the cylindrical surface expressed in in positive topographic relief)), or any other suitable shape.

The anchoring hull may include a head and a tail. The head may have openings. The tail may have one, two or more screw holes. For the purposes of the application, apparatus disclosed as engaging the anchoring hull is engaging openings in the head of the anchoring hull unless stated otherwise.

The anchoring hull may include apparatus and methods described in commonly owned U.S. patent application Ser. No. 12/353,855, filed on Jan. 14, 2009, now U.S. Pat. No. 8,287,538, U.S. patent application Ser. No. 13/043,190, filed on Mar. 8, 2011, now U.S. Pat. No. 8,906,022, and U.S. patent application Ser. No. 13/945,137, filed on Jul. 18, 2013, now U.S. Pat. No. 9,730,739, all of which are hereby incorporated by reference herein in their entireties. The anchoring hull may include any other apparatus and methods known to those skilled in the art.

The anchoring hull may be expandable. The anchoring hull may be an anchoring hull that is not expandable. The anchoring hull may self-expand when deployed. The anchoring hull may expand when rotated. The anchoring hull may be expanded using one or more actuating mechanisms. The anchoring hull may be radially expandable. The anchoring hull may be longitudinally expandable. The anchoring hull, when expanded, may form a mesh cage. The mesh cage may include interconnected cells. The anchoring hull, when expanded, may take on any suitable shape.

The anchoring hull may be implanted in an interior of the bone. The anchoring hull may be implanted retrograde. The anchoring hull may be implanted antegrade. When the anchoring hull is implanted retrograde, the anchoring hull may be expanded in the interior. When the anchoring hull is implanted antegrade, the anchoring hull may be expanded prior to implantation. When the anchoring hull is implanted antegrade, the anchoring hull may be an anchoring hull that is not expandable.

The anchoring hull may be implanted in a cavity formed in the interior. The cavity may be prepared using rongeurs, an osteotome, drills, reamers, cavity preparation devices, or any other apparatus used by those skilled in the art to prepare a cavity. The cavity may be prepared using apparatus and methods described in one or more of commonly owned U.S. patent application Ser. No. 13/009,657, filed on Jan. 19, 2011, now U.S. Pat. No. 8,961,518, U.S. patent application Ser. No. 14/568,301, filed on Dec. 12, 2014, and U.S. patent application Ser. No. 15/439,326, filed on Feb. 22, 2017, all of which are hereby incorporated by reference herein in their entireties.

The anchoring hull may be implanted in the prepared cavity. Implanting the anchoring hull into the prepared cavity may be performed using apparatus and methods described in one or more of commonly owned U.S. patent application Ser. No. 13/043,330, filed on Mar. 8, 2011, U.S. patent application Ser. No. 13/414,695, filed on Mar. 7, 2012, now U.S. Pat. No. 9,498,370, U.S. patent application Ser. No. 15/399,369, filed on Jan. 5, 2017, and U.S. patent application Ser. No. 15/439,326, filed on Feb. 22, 2017, all of which are hereby incorporated by reference herein in their entireties.

The anchoring hull may be used to hold the prosthesis in place on the prepared surface. The anchoring hull may hold the prosthesis in place by receiving screws that are passed through the prosthesis, through a layer of bone positioned between the anchoring hull and the prosthesis, and into the anchoring hull.

The layer of bone may include a void. The layer of bone may include two or more voids. The layer may include no voids.

The layer of bone may be a layer of native bone.

The layer of bone may be a layer comprising one or more of bone cement, allograft, bone graft, cortical bone, glue, composite, processed bone, native bone such as cancellous bone, and any other suitable material.

The anchoring hull may be used to assist in reducing a fracture. The anchoring hull may assist in reducing a fracture by engaging a screw driven through a bone segment. The bone segment may be a first bone segment. The first bone segment may be separated from a second bone segment by a fracture. For the purposes of the application, a first bone segment is separated from a second segment by a fracture when the first bone segment is detached, or partially detached, from the second bone segment by the fracture. The anchoring hull may assist in reducing fracture by engaging two or more screws driven through one, two or more bone segments. The bone segments may not be on a surface of the bone including the articular surface or the prepared surface.

The anchoring hull may be used during a clinical procedure both to assist in fracture reduction and to hold the prosthesis in place.

The anchoring hull may be used to provide healing of a broken bone during a first procedure in which the prosthesis is not prescribed. If, after completion of the first procedure, the prosthesis is prescribed, a practitioner may use the already-in situ anchoring hull to hold the prosthesis in place.

For example, a practitioner, in the first procedure, may make a first incision in soft tissue covering a bone, anchoring hull the anchoring hull in an interior of the bone, and drive screws through fractured bone and into the anchoring hull. Fracture reduction performed during the first procedure may be performed using apparatus and methods described in one or more of commonly owned U.S. patent application Ser. No. 13/043,330, filed on Mar. 8, 2011, U.S. patent application Ser. No. 13/414,695, filed on Mar. 7, 2012, now U.S. Pat. No. 9,498,370, U.S. patent application Ser. No. 15/399,369, filed on Jan. 5, 2017, and U.S. patent application Ser. No. 15/439,326, filed on Feb. 22, 2017, all of which are hereby incorporated by reference herein in their entireties.

The practitioner may then close the first incision. In the event that a condition of the patient worsens, the patient does not respond to the therapy provided, the patient develops arthritis, the patient suffers other damage to the bone, the practitioner may then make a second incision, in a second procedure in which native articular surface is removed, and use the anchoring hull as a support for an implanted articular surface prosthesis.

The prosthesis may have: a first face shaped to conform to a native articular bone surface and defining a perimeter; and a second face shaped to conform to a bone surface prepared for receiving the prosthesis; and defining two or more screw-holes adjacent the perimeter. The prosthesis may define, other than the screw-holes, no anchor pass-through.

The prosthesis may include a thickness between the first face and the second face; a normal to the first face pointing in a first direction; a normal to the second face pointing in a second direction; and the first and second directions may point in directions that diverge from each other.

The prosthesis may be a prosthesis that does not include an anchoring stem. The prosthesis may be a prosthesis that does not include a fixture for receiving an anchoring stem.

The prosthesis may be a prosthesis that does not include an anchoring stem that extends from the second face. The prosthesis may be a prosthesis that does not include an anchoring stem that extends only from the second face.

The two or more holes may be sized to receive a screw. The two or more holes may be sized to receive a screw having a length in the range 10 mm to 120 mm. The two or more holes may be sized to receive a screw that has a diameter in the range 1.1 mm to 10 mm.

Each of the two or more holes may define a first opening on the first face and a second opening on the second face. The first opening may be larger than the second opening. The second face may be smooth. A smooth second face may not define a stem for coupling to the anchoring hull.

The prosthesis may be dome-shaped.

The prosthesis may define a truncated sphere.

The first surface may have a topography that includes one or more valleys and one or more hills.

The two or more holes may include: a first hole having a diameter and a centroid that is at a first distance from the perimeter; a second hole having a diameter and a centroid that is at a second distance from the perimeter; a third hole having a diameter and a centroid that is at a third distance from the perimeter.

All of the first, second and third distances may be a distance that is no less than 1.5 times the diameter.

A one of the first, second and third distances may be a distance that is no more than 1.5 times the diameter.

All of the first, second and third distances may be distances that are not more than 1.5 times the diameter.

A one of the first, second and third distances may be a distance that is no more than 2 times the diameter.

All of the first, second and third distances may be distances that are not more than 2 times the diameter.

A one of the first, second and third distances may be a distance that is no more than 2.5 times the diameter.

All of the first, second and third distances may be distances that are not more than 2.5 times the diameter.

A one of the first, second and third distances may be a distance that is no more than 3 times the diameter.

All of the first, second and third distances may be a distances that is not more than 3 times the diameter.

The two or more holes may include: a first hole having a diameter and a centroid that is at a first distance from the perimeter; a second hole having a diameter and a centroid that is at a second distance from the perimeter; a third hole having a diameter and a centroid that is at a third distance from the perimeter.

All of the first, second and third distances may be a distance that is no less than the diameter.

A one of the first, second and third distances may be a distance that is no more than the diameter.

All of the first, second and third distances may be distances that are not more than the diameter.

A one of the first, second and third distances may be a distance that is no more than 1.5 times the diameter.

All of the first, second and third distances may be distances that are not more than 1.5 times the diameter.

A one of the first, second and third distances may be a distance that is no more than 2 times the diameter.

All of the first, second and third distances may be distances that are not more than 2 times the diameter.

A one of the first, second and third distances may be a distance that is no more than 2.5 times the diameter.

All of the first, second and third distances may be distances that are not more than 2.5 times the diameter.

A one of the first, second and third distances may be a distance that is no more than 3 times the diameter.

All of the first, second and third distances may be distances that are not more than 3 times the diameter.

The two or more holes may be positioned, along a path that contours the perimeter, at uniform offsets from each other.

The anchoring hull may define a plurality of openings. The prosthesis and the anchoring hull may be configured to be fixed together by screws. The prosthesis and the anchoring hull may be configured to be fixed together by one or more tension-applying members such as sutures. Direct physical contact between the prosthesis and the anchoring hull, when therapeutically in situ, may be a contact at which the anchoring hull does not retain the prosthesis.

The prosthesis may be a prosthesis that is not configured to mate with the anchoring hull. The prosthesis may be a prosthesis that is not configured to snap into the anchoring hull. Abutment of the prosthesis against the anchoring hull may be an abutment that does not rigidly couple the prosthesis to the anchoring hull.

The plurality of openings may be sized to receive screws having a length in the range 10 mm to 120 mm and a diameter in the range 1.1 mm and 10 mm.

The plurality of openings defined by the anchoring hull may have a first number of openings. The two or more holes defined by the prosthesis have a second number of openings. The first number may be at least ten times the second number.

The apparatus may include first means for performing a function of native bone tissue; second means for anchoring the first means to the bone; and third means for fastening the first means to the second means. The first and second means may be disposed on opposite sides of a layer of bone and, exclusive of the bone and the third means, have no structural interconnection therebetween. The first and second means may be disposed on opposite sides of the layer of bone and, exclusive non-native elements, have no structural interconnection therebetween.

The methods may include implanting the anchoring hull in an interior of the bone. The methods may include removing native articular bone to provide a prepared surface upon which to seat the prosthesis. The methods may include seating the prosthesis on the prepared surface in a position in which the prosthesis is entirely spaced apart from the anchoring hull by a layer of bone.

The layer of bone may be a layer of native bone.

The layer of bone may be a layer of both native bone and one or more of bone cement, allograft, bone graft, or any other synthetic or natural substance.

In the implanting, the anchoring hull may be placed in the bone within reach of screws that are to be driven through the prosthesis and into the anchoring hull. However, in the implanting, there may be no predetermined orientation of the anchoring hull that is required to provide correspondence between prosthesis holes and anchoring hull openings, other than placing of the anchoring hull nearby the prosthesis in the interior. Thus, the implanting may not limit a practitioner to one or more areas on the bone for performing the removing of the native articular surface bone, and the seating may not limit a practitioner to one or more areas in the interior of the bone for performing the implanting of the anchoring hull.

In the placing, there may be no predetermined orientation of the prosthesis on the prepared surface that is required to provide correspondence between prosthesis holes and anchoring hull openings.

The methods may include using a jig to seat the prosthesis on the prepared surface.

The methods may include seating the prosthesis on the prepared surface without using a jig.

The removing the native articular bone may include removing tissue adjacent the articular bone.

The layer of bone may be defined, on a side of the layer, by the prepared surface.

The side may be a first side.

The methods may include, prior to the implanting, forming a cavity in the interior. The cavity may define a second side of the layer. The second side may be opposite the first side. The second side may be separated from the first side by the layer.

The implanting may include positioning the anchoring hull adjacent the second side of the layer. The anchoring hull may be positioned adjacent the second side during the implanting when the implanting is performed retrograde. The anchoring hull may be positioned adjacent the second side during the implanting when the implanting is performed antegrade.

When the anchoring hull is positioned in the interior antegrade, the methods may include, after the implanting, arranging native bone fragments on an outer surface of the anchoring hull, and, then, performing the preparing. The arranging may define the layer of bone. The arranging may position a second side of the layer adjacent the anchoring hull.

The implanting may include placing the anchoring hull in abutment with the second side of the layer. The anchoring hull may be positioned adjacent the second side during the implanting when the implanting is performed retrograde. The anchoring hull may be positioned adjacent the second side during the implanting when the implanting is performed antegrade.

When the anchoring hull is positioned in the interior antegrade, the arranging may define the layer of bone, and the second side of the layer may be positioned in abutment with the anchoring hull.

The implanting may be an implanting without penetrating the layer during the implanting.

The implanting may be an implanting without penetrating the layer when the implanting is performed retrograde.

The seating may be a seating without penetrating the layer.

The implanting may be an implanting that is without crossing the first side of the layer.

The implanting may not cross the first side of the layer when the implanting is performed retrograde.

The seating may be a seating that is without crossing the first side of the layer.

The implanting may include placing the anchoring hull adjacent a second side of the layer, the second side opposite the first side and separated from the first side by the layer. The placing the hull adjacent a second side of the layer, the second side opposite the first side and separated from the first side by the layer, may be without crossing the second side of the layer.

The implanting may include placing the anchoring hull adjacent the second side of the layer. The implanting may include placing the hull adjacent the second side of the layer and the seating may be without crossing the second side of the layer.

The implanting and the seating may together sandwich the layer of bone between a bottom surface of the prosthesis and an outer surface of the anchoring hull.

When the anchoring hull is implanted antegrade, the implanting, the arranging of the bone segments, and the seating may together sandwich the layer of bone between a bottom surface of the prosthesis and an outer surface of the anchoring hull.

The seating may be a seating that is without a touching of a bottom surface of the prosthesis to the anchoring hull.

The preparing may be performed before the implanting. The preparing may be performed before the seating.

The implanting may be performed before the preparing. The implanting may be performed before the removing. The implanting may be performed before the seating.

The seating may be performed before the implanting.

The methods may include driving a screw through a hole in the prosthesis, through the layer of bone and into the anchoring hull. The driving may be a driving that is included in a driving of a plurality of screws through a plurality of holes in the prosthesis. Each of the screws may correspond to one of the holes, penetrate the layer and lodge in the anchoring hull. Lodging in the anchoring hull may include terminating in the anchoring hull. Lodging in the anchoring hull may include terminating after exiting the anchoring hull.

The methods may include not providing, aside from the driving, a therapeutic fixation that bridges between the prosthesis and the anchoring hull.

The driving may be a driving that does not abut the prosthesis against the anchoring hull.

The methods may include driving a screw through a hole in the prosthesis, through the layer of bone and into an opening in the anchoring hull to secure the prosthesis to the anchoring hull. The driving may be a driving that does not abut the prosthesis against the anchoring hull.

The methods may include driving a screw through the prosthesis hole at an angle (α′) to a central axis of the prosthesis hole. The screw may have a screw head that is round to seat into a counter-sunk, tapered, or contoured entry into the screw-hole. The hole may be shaped to receive the head of the screw at a fixed angle or at a range of angles. The range may have a maximum angle (α).

The methods may include selecting an angle for driving the screw through the prosthesis hole from a continuous range of 0°-65° away from a central axis of the prosthesis hole. The methods may include selecting an angle for driving the screw through the prosthesis hole from a continuous range of 0°-80° away from a central axis of the prosthesis hole.

The opening may be a first opening. The screw may have a screw head. The screw may have a screw tip. The screw may have a first screw length that extends between the screw head and the screw tip. The methods may include: after driving the screw through the first opening, driving the screw through a volume defined by the anchoring hull, through a second opening in the anchoring hull. The volume defined by the anchoring hull may be a volume inside a head of the anchoring hull. The methods may include seating the screw head in the prosthesis hole such that a second length of the screw extending between the first and second opening is not less than half of the first length.

The seating may include seating the screw head in the prosthesis hole such that a third length of the screw extending between the screw head and the first opening is not less than one fifth of the first length.

The methods may include engaging the screw tip with cortical bone. Engaging the screw tip with cortical bone may reduce a fracture in the bone.

TABLE 2
Illustrative ratios of lengths (L₁ = screw length, L₂ = hull
traverse length, L₃ = screw head to hull-entry length, L₄ =
hull-exit to screw tip length). Lower and upper ratio ranges may be
considered as a pair. Individual lower and upper ratio limits may be
considered as one-sided limits, e.g., the lower limit 0.05 may be
considered a one-sided limit, meaning no less than 0.05, or 0.05
or greater than 0.05. Sub ranges defined by a lower limit selected
from the Table and an upper limit selected from the Table, even if
not on the same row of the Table, are included.

| L₂:L₁ | | L₃:L₂ | | L₂:L₃ | | | |
|---|---|---|---|---|---|---|---|
| (range limits, | | (For L₃ ≤ L₂; | | (For L₂ ≤ L₃; | | L₄:L₂ | |
| endpoints | | range limits, | | range limits, | | (range limits, | |
| inclusive) | | endpoints | | endpoints | | endpoints | |
| Low- | | inclusive) | | inclusive) | | inclusive) | |
| er | Upper | Lower | Upper | Lower | Upper | Lower | Upper |
| 0.0 | 0.05 | 0.0 | 0.05 | 0.0 | 0.05 | 0.0 | 0.05 |
| 0.05 | 0.10 | 0.05 | 0.10 | 0.05 | 0.10 | 0.05 | 0.10 |
| 0.10 | 0.15 | 0.10 | 0.15 | 0.10 | 0.15 | 0.10 | 0.15 |
| 0.15 | 0.20 | 0.15 | 0.20 | 0.15 | 0.20 | 0.15 | 0.20 |
| 0.20 | 0.25 | 0.20 | 0.25 | 0.20 | 0.25 | 0.20 | 0.25 |
| 0.25 | 0.30 | 0.25 | 0.30 | 0.25 | 0.30 | 0.25 | 0.30 |
| 0.30 | 0.35 | 0.30 | 0.35 | 0.30 | 0.35 | 0.30 | 0.35 |
| 0.35 | 0.40 | 0.35 | 0.40 | 0.35 | 0.40 | 0.35 | 0.40 |
| 0.40 | 0.45 | 0.40 | 0.45 | 0.40 | 0.45 | 0.40 | 0.45 |
| 0.45 | 0.50 | 0.45 | 0.50 | 0.45 | 0.50 | 0.45 | 0.50 |
| 0.50 | 0.55 | 0.50 | 0.55 | 0.50 | 0.55 | 0.50 | 0.55 |
| 0.55 | 0.60 | 0.55 | 0.60 | 0.55 | 0.60 | 0.55 | 0.60 |
| 0.60 | 0.65 | 0.60 | 0.65 | 0.60 | 0.65 | 0.60 | 0.65 |
| 0.65 | 0.70 | 0.65 | 0.70 | 0.65 | 0.70 | 0.65 | 0.70 |
| 0.70 | 0.75 | 0.70 | 0.75 | 0.70 | 0.75 | 0.70 | 0.75 |
| 0.75 | 0.80 | 0.75 | 0.80 | 0.75 | 0.80 | 0.75 | 0.80 |
| 0.80 | 0.85 | 0.80 | 0.85 | 0.80 | 0.85 | 0.80 | 0.85 |
| 0.85 | 0.90 | 0.85 | 0.90 | 0.85 | 0.90 | 0.85 | 0.90 |
| 0.90 | 0.95 | 0.90 | 0.95 | 0.90 | 0.95 | 0.90 | 0.95 |
| 0.95 | 1.00 | 0.95 | 1.00 | 0.95 | 1.00 | 0.95 | 1.00 |

The length of L₄ may be zero. L₃ may be greater than L₂.

The methods may include driving a second screw through a second hole in the prosthesis, through the layer of bone, and into a second opening in the anchoring hull.

The methods may be methods in which there is no therapeutic fixation member bridging from the prosthesis to the anchoring hull other than the first and second screws.

The methods may include driving a third screw through a third hole in the prosthesis, through the layer of bone and into a third opening in the anchoring hull.

The methods may be methods in which there is no therapeutic bridge running from the prosthesis to the anchoring hull other than the first, second and third screws.

The methods may include selecting a location that is on the bone, but not on the prepared surface, for driving a screw through the bone and into the anchoring hull. The selecting may be performed without registering the location to a particular opening, of the many openings, in the anchoring hull. The location may be on epiphyseal bone. The location may be on diaphyseal bone. The location may be on metaphyseal bone. The location may be on a first segment of bone that is separated from a second segment of bone by a fracture.

The selecting may include selecting the location from a plurality of locations that are distributed continuously within a region of the bone. The bone may be a humerus. The plurality of locations may include a lateral surface of the humerus. The plurality of locations may include a medial surface of the humerus. The plurality of locations may include a proximal surface of the humerus. The plurality of locations may include two or more of the lateral surface, the medial surface and the proximal surface. The plurality of locations may define the humerus in its entirety.

The selecting may be a selecting that does not include selecting from predetermined set points.

The selected location may be on a bone surface. The bone surface may define a surface normal. The methods may include selecting an angle, from angles in a continuous range between 0° and 90° relative to the bone surface normal, for performing the driving. The methods may include selecting an angle, from angles in a continuous range between 0° and 80° relative to the bone surface normal, for performing the driving. The methods may include driving a screw through the location at the selected angle and into the anchoring hull.

The methods may include, after the identifying, selecting a screw for performing the driving from screws having lengths ranging from 10 mm to 120 mm.

The methods may include driving the selected screw through the location and into the anchoring hull. The driving may performed without using a jig. The driving may be performed without using a jig to register the screw with an opening in the anchoring hull.

The methods may include driving the screw through a first opening defined by the anchoring hull. The methods may include driving the screw through a volume defined by the anchoring hull. The methods may include driving the screw through a second opening defined by the anchoring hull. The methods may include engaging a tip of the screw with cortical bone after driving the tip through the second opening.

The bone may be fractured. The driving of a screw through the selected location and into the anchoring hull may reduce the fracture. Prior to driving the screw, the fracture may be reduced by a practitioner manually. Prior to driving the screw, the fracture may be reduced with one or more k-wires. Prior to driving the screw, the fracture may be reduced using one or more sutures.

The bone may include a fractured tuberosity. The fractured tuberosity may be a greater tuberosity. The fractured tuberosity may be a lesser tuberosity. The driving may include driving the screw through the fractured tuberosity. The driving of the screw may reduce the fracture of the tuberosity. The methods may include driving two or more screws through the fractured tuberosity and into the anchoring hull. The driving of the two or more screws may reduce the fracture of the tuberosity. The methods may include driving two or more screws through both the greater and the lesser tuberosities to reduce fractures in the tuberosities.

The methods may include selecting a location that is on the bone, but not on the prepared surface, for driving a screw into the anchoring hull. The methods may include driving a k-wire through the location, the driving including aiming the k-wire at the anchoring hull without aligning the k-wire with a designated hole in the anchoring hull. The methods may include driving the screw through the location and into the anchoring hull. The location may be on a first segment of bone separated from a second segment of bone by a fracture of the bone. The selecting may include selecting a plurality of locations that are continuously distributed within a region of the bone. The location may have a bone surface normal. The methods may include selecting an angle for performing the driving of the k-wire, and the driving of the screw, from a plurality of angles in a continuous range between 0° and 90° relative to the bone surface normal. The methods may include selecting an angle for performing the driving of the k-wire, and the driving of the screw, from a plurality of angles in a continuous range between 0° and 80° relative to the bone surface normal. The continuous range of angles may be constrained by a diameter of the anchoring hull.

The driving the k-wire may be performed without using a jig. The driving the k-wire may be performed without using a jig to register the k-wire to an opening in the anchoring hull.

The driving the screw may include positioning a bore of the screw over the k-wire. The driving the screw may include advancing the screw along the k-wire and into the anchoring hull.

The methods may include driving the screw over the k-wire and into the anchoring hull. The methods may include driving the screw through a first opening defined by the anchoring hull. The methods may include driving the screw through a volume defined by the anchoring hull. The methods may include driving the screw through a second opening defined by the anchoring hull. The methods may include engaging a tip of the screw with cortical bone after driving the tip through the second opening.

The bone may include a first segment and a second segment separated from the first segment by a fracture. The methods may include driving a first screw through the first segment and into the anchoring hull. The methods may include driving a second screw through the second segment and into the anchoring hull. The driving the first screw may reduce the fracture. The driving the second screw may reduce the fracture. The methods may include providing a first incision in soft tissue covering the bone, then, performing the implanting and the driving of the first screw and the second screw. The methods may include closing the first incision, then, providing a second incision in soft tissue covering the bone, and, then, performing the removing and the seating.

The first incision may be performed as therapy for a first diagnosis. The second incision may be performed a therapy for a second diagnosis. The second diagnosis may be rendered after the closing of the first incision.

The methods may include, after the seating, driving a screw through a hole in the prosthesis, through the layer of bone, into an opening of the anchoring hull and into a volume defined by the anchoring hull. The driving may include displacing bone matter accumulated inside the anchoring hull after the providing of the first incision and before the providing of the second incision. The bone may be a humerus. The bone may include a tuberosity. The fracture may be a fracture of the tuberosity.

The bone may include a segment. The methods may include driving a screw through the segment and into the anchoring hull. The driving first screw may reduce a fracture in the bone. The methods may include providing a first incision in soft tissue covering the bone, then, performing the implanting and the driving of the screw. The methods may include closing the first incision, then, providing a second incision in soft tissue covering the bone, and, then, performing the removing and the seating.

The first incision may be performed as therapy for a first diagnosis. The second incision may be performed a therapy for a second diagnosis. The second diagnosis may be rendered after the closing of the first incision.

The methods may include, after the seating, driving a screw through a hole in the prosthesis, through the layer of bone and into an opening of the anchoring hull. The driving may include displacing bone matter accumulated inside the anchoring hull after the providing of the first incision and before the providing of the second incision. The bone may be a humerus. The bone may include a tuberosity. The fracture may be a fracture of the tuberosity.

The methods may include selecting an area on the native articular bone surface for the preparing. The selecting may be a selecting that does not include registering a feature of the prosthesis to a particular predetermined feature of the anchoring hull. The feature of the prosthesis may be a hole. The particular predetermined feature may be one of a plurality of openings in the anchoring hull.

Because the prosthesis is not necessarily registered to a particular feature of the anchoring hull, a practitioner may select the area for the preparing from a plurality of areas that are distributed continuously along the articular surface. The area selected by the practitioner may not be required based on the position or orientation of the anchoring hull in the interior.

The native articular surface may be dome-shaped. The native articular surface may include a first apex. The prepared surface may include a second apex. The seating may be performed without requiring that the second apex be positioned at a predetermined distance from a position of the first apex. The seating may include selecting may include selecting an angle of offset between the first apex and the second apex from a plurality of angles in a continuous range between 0° and 15°.

The implanting may be performed retrograde. The seating may be performed before the implanting. The seating may be performed after the implanting. The methods may include selecting a location for preparing an access hole on a surface of the bone. The location may be a location that is not on native articular surface, and is not on prepared surface. The selecting may be a selecting that is performed without registering an opening in the anchoring hull to a hole in the prosthesis.

The methods may include preparing the access hole. The methods may include advancing a cavity preparation instrument through the access hole. The methods may include forming the cavity in the interior using the instrument. The cavity may define a side of the layer. The methods may include advancing the anchoring hull through the hole along a central axis of the hole. The methods may include placing the anchoring hull adjacent the side of the layer. The methods may include placing the anchoring hull adjacent in abutment with the side of the layer. The methods may include expanding, in the interior, a plurality of openings in a head of the anchoring hull.

The methods may include accessing a first portion of the bone for the placing. The methods may include accessing a second portion of the bone spaced apart from the first portion of the bone for performing the retrograde implanting. The bone may be a proximal humerus. The access hole may be prepared on a lateral surface of the bone.

The implanting may be performed antegrade. When the anchoring hull is expandable, the methods may include expanding a plurality of openings in a head of the anchoring hull prior to placing the anchoring hull in the interior. The methods may include, after the implanting, arranging native bone fragments on an outer surface of the anchoring hull, and, then, performing the preparing. The arranging may define the layer of bone. The arranging may define a first side of the layer of bone. After the arranging, the native bone fragments may be prepared to receive the prosthesis. The preparation may include removing native articular bone. The prepared surface may define a second side of the layer of bone.

The methods may include selecting an implanting technique for the implanting. The implanting technique may be antegrade or retrograde.

The methods may include selecting a location in the interior for the implanting of the anchoring hull. The position may be selected without registering a feature of the prosthesis to a feature of the anchoring hull. The position may be selected from a plurality of locations that are distributed continuously in the interior of the bone. A first location may be more medial, lateral, proximal or distal relative to a second position.

The methods may include selecting a thickness of the layer of native bone. The selecting the thickness may include both selecting an amount of the native articular bone surface to be removed and selecting a location in the interior for implanting the anchoring hull.

The methods may include selecting a location in the interior for the implanting. The selecting may include selecting between a first position and a second position that is located in the interior medial the first position. The selecting may be a selecting that does not including registering a feature of the anchoring hull with a feature of the prosthesis. The selecting may be a selecting that does not register one opening included in a plurality of openings in the anchoring hull with a hole in the prosthesis.

The methods may include selecting a location in the interior for the implanting. The selecting may include selecting between a first position and a second position that is located in the interior proximal the first position. The selecting may be a selecting that does not include registering a feature of the anchoring hull with a feature of the prosthesis. The selecting may be a selecting that does not register one opening included in a plurality of openings in the anchoring hull with a hole in the prosthesis.

The prosthesis may include two or more holes. The methods may include driving screws through each of the two or more holes, through the layer of bone and into openings in the anchoring hull. The methods may include leaving twenty or more of the openings unoccupied by screws.

The methods may include providing fifty or more openings in the anchoring hull. The methods may include engaging twelve or fewer of the openings with screws.

The anchoring hull may include a mesh cage. The methods may include driving a screw through a first hole in the prosthesis and into the mesh cage. The methods may include driving a second screw through a second hole in the prosthesis and into the mesh cage. The screws may anchor the prosthesis to the cage. The methods may be a method in which there is no therapeutic fixation, other than the screws, that bridges from the prosthesis to the cage.

The methods may include implanting, in an interior of the bone, the anchoring hull. The methods may include preparing an articular surface of the bone for receiving the prosthesis. The methods may include placing the prosthesis on the prepared surface. The prosthesis may abut the anchoring hull. The abutment may be an abutment that does not provide engagement between the prosthesis and the hull. The methods may include fixing the prosthesis to the anchoring hull without using a snap-fit feature. The methods may include fixing the prosthesis to the anchoring hull without using a press-fit feature. The methods may include fixing the prosthesis to the anchoring hull without threadingly engaging the prosthesis with the anchoring hull other than by the use of screws.

The methods may include driving a screw through two or more holes defined by the prosthesis and into the anchoring hull. The methods may be methods in which, other than a screw, there is no therapeutic fixation between the prosthesis and the anchoring hull.

The methods may include, after the seating, circumferentially rotating the prosthesis about an axis normal to the prosthesis and at an apex of the prepared surface when the prosthesis defines a spherical shape.

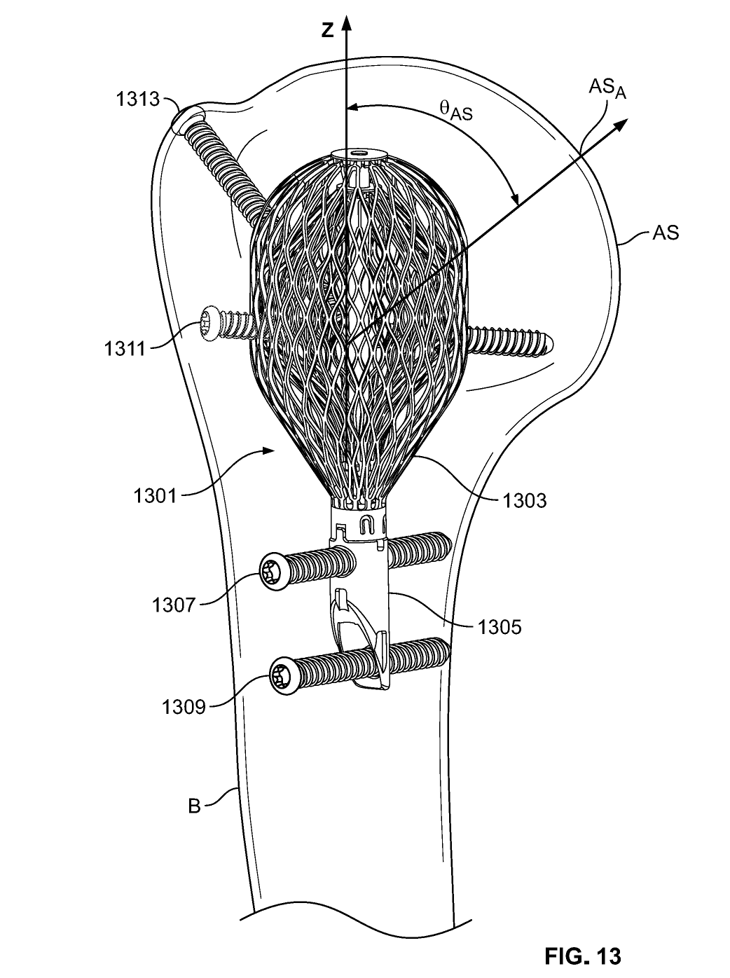

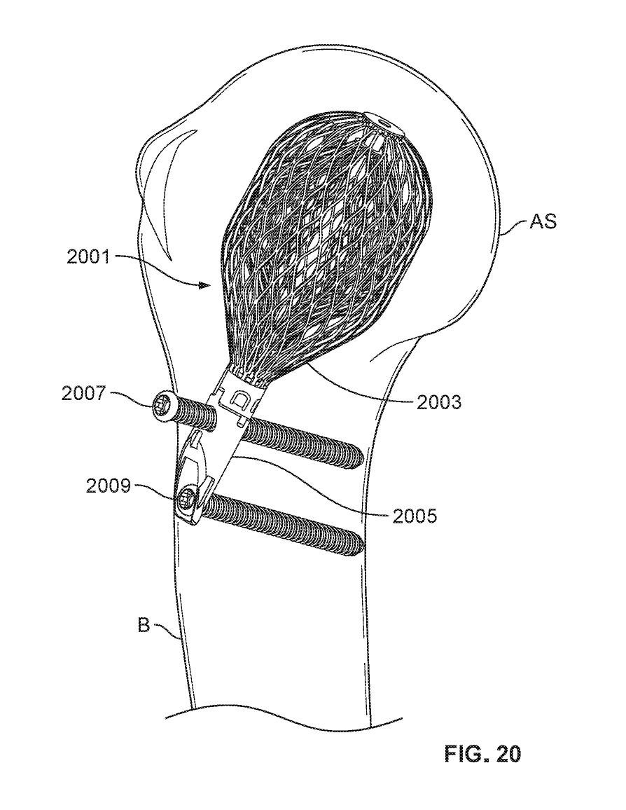

The steps of illustrative methods, such as methods illustrated in FIGS. 13-19 and methods illustrated in FIGS. 20-26, may be performed in an order other than the order shown and/or described herein. Some embodiments may omit steps shown and/or described in connection with the illustrative methods. Some embodiments may include steps that are neither shown nor described in connection with the illustrative methods such as methods illustrated in FIGS. 13-19 and FIGS. 20-26. Illustrative method steps may be combined. For example, one illustrative method may include steps shown in connection with another illustrative method.

Some embodiments may omit features shown and/or described in connection with the illustrative apparatus. Some embodiments may include features that are neither shown nor described in connection with the illustrative apparatus. Features of illustrative apparatus may be combined. For example, one illustrative embodiment may include features shown in connection with another illustrative embodiment.

Embodiments may involve some or all of the features of the illustrative apparatus and/or some or all of the steps of the illustrative methods.

The illustrative apparatus and therapeutic scenarios will now be described now with reference to the accompanying drawings in the Figures, which form a part hereof. It is to be understood that other embodiments may be utilized and that structural, functional and procedural modifications may be made without departing from the scope and spirit of the present disclosure.

For the sake of clarity, figures may illustrate therapeutic treatment of bones without showing fractures.

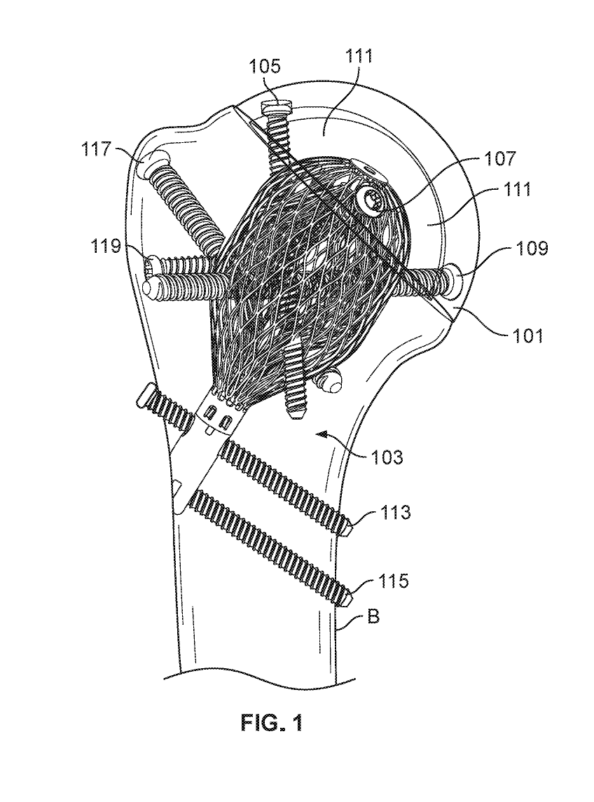

FIG. 1 shows illustrative prosthesis 101 seated on bone B. Bone B is illustrated as being a humerus (and, in particular, the proximal humerus). The apparatus and methods may be practiced on bones other than the humerus. Prosthesis 101 may be seated on a surface of bone B that was prepared by removing native articular bone surface from bone B. FIG. 1 also shows illustrative anchoring hull 103 implanted retrograde in an interior of bone B. In FIG. 1, prosthesis 101 is illustrated as transparent to show layer of bone 111 positioned between prosthesis 101 and anchoring hull 103. Layer of bone 111 separates the entire prosthesis 101 from anchoring hull 103 so that there is no abutment between prosthesis 101 and anchoring hull 103.

Screws 105, 107 and 109 are shown extending from holes in prosthesis 101 and into first openings in anchoring hull 103. Screws 105, 107 and 109 are shown passing through a volume defined by anchoring hull 103, engaging second openings in anchoring hull 103 and extending away from anchoring hull 103. One or more tips of screws 105, 107 and 109 may engage cortical bone on bone B. The engagement of a tip of a screw with cortical bone on bone B may reduce a fracture in bone B (not shown).

In FIG. 1, screws 113 and 115 are shown engaging a tail of anchoring hull 103 to a diaphyseal shaft of bone B.

Screws 117 and 119 are shown passing through a tuberosity of bone B and into anchoring hull 103. Screws 117 and 119 illustrate placement of screws by a practitioner to reduce a fracture in a tuberosity of bone B (fracture not shown).

FIG. 1, and all subsequent FIGS. showing screws passing through one or more prosthesis holes and/or a surface of the bone, illustrate possible trajectories of screws. Any other suitable trajectories may be used to drive screws through the prosthesis and to drive screws through bone B and into the anchoring hull. The screw trajectories may be selected to reduce one or more fractures.

The implanting of anchoring hull 103 in bone B, and the fixing of the tuberosity to anchoring hull 103 by screws 113 and 115, may be performed during a clinical procedure that includes placing prosthesis 101 on bone B.

The implanting of anchoring hull 101 into bone B, and the fixing of the tuberosity to anchoring hull 103 by screws 13 and 115, may be performed during a first procedure in which the prosthesis is not placed on bone B. The prosthesis may be placed on bone B during a second procedure performed at later point in time after completion of the first procedure.

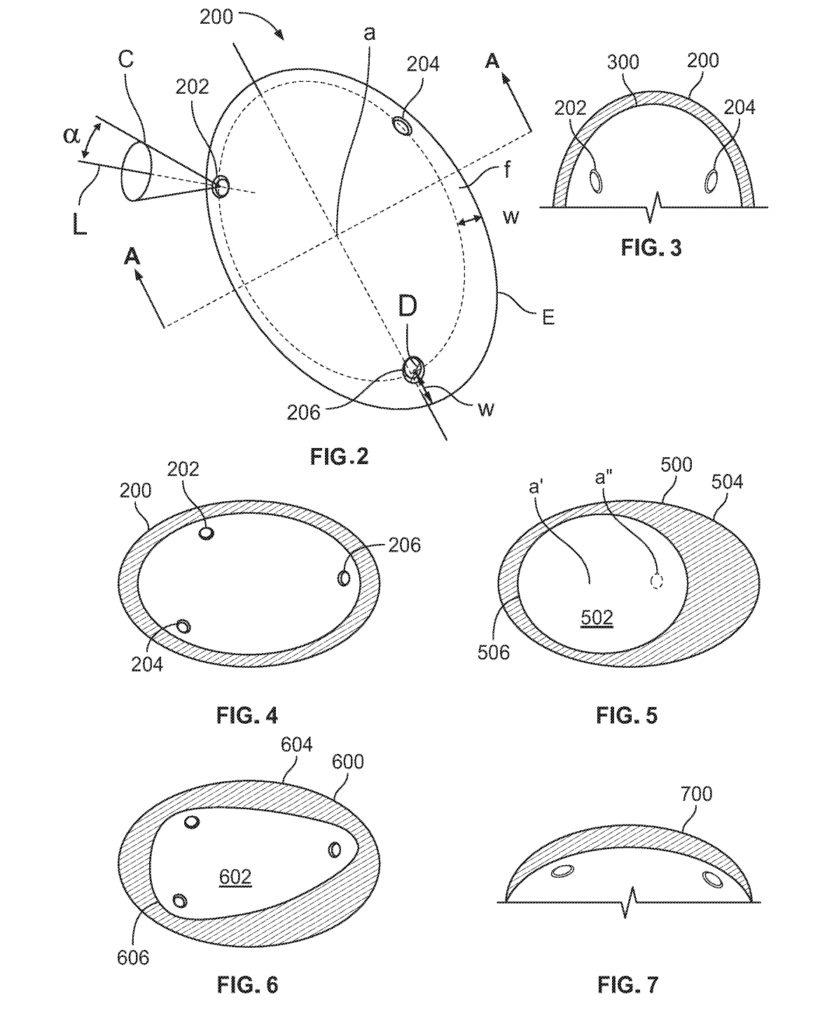

FIG. 2 shows schematically illustrative prosthesis 200. Perimeter region P has a tracks along edge E at a distance from edge E. Screw holes 202, 204, and 206 have diameters D. Screw holes 202, 204 and 206 have centroids disposed towards apex a away from edge E. Perimeter region P is shown has having a width W. W may have any suitable value. Table 3 shows illustrative values of W in terms of D.

TABLE 3
Illustrative values of W in terms of D (W:D). Lower and upper
ratio ranges may be considered as a pair. Individual lower and
upper ratio limits may be considered as one-sided limits, e.g.,
the lower limit 0.7 may be considered a one-sided limit, meaning
no less than 0.7, or 0.7 or greater than 0.7. Sub ranges defined by
a lower limit selected from the Table and an upper limit selected
from the Table, even if not on the same row of the Table, are included.
Illustrative values of W in terms of D
(W:D; range limits, endpoints inclusive)

| Lower | Upper |
|---|---|
| 0.7 | 0.8 |
| 0.8 | 0.9 |
| 0.9 | 1 |
| 1 | 1.1 |
| 1.1 | 1.2 |
| 1.2 | 1.3 |
| 1.3 | 1.4 |
| 1.4 | 1.5 |
| 1.5 | 1.6 |
| 1.6 | 1.7 |
| 1.7 | 1.8 |
| 1.8 | 1.9 |
| 1.9 | 2 |
| 2 | 2.1 |
| 2.1 | 2.2 |
| 2.2 | 2.3 |
| 2.3 | 2.4 |
| 2.4 | 2.5 |
| 2.5 | 2.6 |
| 2.6 | 2.7 |
| 2.7 | 2.8 |
| 2.8 | 2.9 |
| 2.9 | 3 |
| 3 | 3.1 |
| 3.1 | 3.2 |
| 3.2 | 3.3 |
| 3.3 | 3.4 |
| 3.4 | 3.5 |
| 3.5 | 3.6 |
| 3.6 | 3.7 |
| 3.7 | 3.8 |
| 3.8 | 3.9 |
| 3.9 | 4 |
| 4 | 4.1 |
| 4.1 | 4.2 |
| 4.2 | 4.3 |
| 4.3 | 4.4 |
| 4.4 | 4.5 |
| 4.5 | 4.6 |
| 4.6 | 4.7 |
| 4.7 | 4.8 |
| 4.8 | 4.9 |
| 4.9 | — |

Theoretical cone C shows a range of orientations at which a screw may be inserted through a hole such as 202. Cone C has an angle α away from central axis L of hole 202. α is a maximum angle away from L that a screw may be inserted into hole 202 so that the screw's trajectory will intersect with the anchoring hull interior the bone and, without prior alignment of a particular anchoring hull opening, engage an opening. Table 4 shows illustrative values of angle α.

TABLE 4
Illustrative values of angle α.
Illustrative values of angle α (degrees of arc)

0
1
2
3
4
5
6
7
8
9
10
11
12
13
14
15
16
17
18
19
20
21
22
23
24
25
26
27
28
29
30
31
32
33
34
35
36
37
38
39
40
41
42
43
44
45
46
47
48
49
50
51
52
53
54
55
56
57
58
59
60
61
62
63
64
65
66
67
68
69
70
71
72
73
74
75
76
77
78
79
80

Hole 202 may be rounded out or countersunk sufficiently to receive a screw head flush with, or below, replacement articular surface, for any screw angle α′ that is in the continuous range of screw angles 0≤α′≤α.

Holes 202, 204 and 206 may be distributed at uniform angles about apex a. This may avoid or reduce stress concentrations in the prosthesis.

FIG. 3 shows a view of prosthesis 200 along lines A-A. Seating surface 300 is visible. Prosthesis 200 may have outer diameter δ. Diameter δ may be a major elliptical axis. Diameter δ may be a minor elliptical axis. For dome-shaped prostheses, diameter δ may be the only diameter. Prosthesis 200 may have height γ. Table 5 shows illustrative values of diameter δ.

TABLE 5
Illustrative values of diameter δ. Lower and upper ratio
ranges may be considered as a pair. Individual lower and
upper ratio limits may be considered as one-sided limits,
e.g., the lower limit 6 may be considered a one-sided limit,
meaning no less than 6, or 6 or greater than 6. Sub ranges
defined by a lower limit selected from the Table and an upper
limit selected from the Table, even if not on the same row of
the Table, are included.
Illustrative values of diameter δ, millimeters
(range limits, endpoints inclusive)

| Lower | Upper |
|---|---|
| 6 | 8 |
| 8 | 10 |
| 10 | 12 |
| 12 | 14 |
| 14 | 16 |
| 16 | 18 |
| 18 | 20 |
| 20 | 22 |
| 22 | 24 |
| 24 | 26 |
| 26 | 28 |
| 28 | 30 |
| 30 | 32 |
| 32 | 34 |
| 34 | 36 |
| 36 | 38 |
| 38 | 40 |
| 40 | 42 |
| 42 | 44 |
| 44 | 46 |
| 46 | 48 |
| 48 | 50 |
| 50 | 52 |
| 52 | 54 |
| 54 | 56 |
| 56 | 58 |
| 58 | 60 |
| 60 | 62 |
| 62 | 64 |
| 64 | 66 |
| 66 | 68 |
| 68 | 70 |
| 70 | 72 |
| 72 | 74 |
| 74 | 76 |
| 76 | 78 |
| 78 | 80 |
| 80 | 82 |
| 82 | 84 |
| 84 | 86 |
| 86 | 88 |
| 88 | 90 |
| 90 | 92 |
| 92 | 94 |
| 94 | 96 |
| 96 | 98 |
| 98 | 100 |
| 100 | 102 |
| 102 | 104 |
| 104 | 106 |
| 106 | 108 |
| 108 | 110 |
| 110 | 112 |
| 112 | 114 |
| 114 | 116 |
| 116 | 118 |
| 118 | 120 |
| 120 | |

Table 6 shows illustrative values of height γ.

TABLE 6
Illustrative values of height γ. Lower and upper ratio
ranges may be considered as a pair. Individual lower and
upper ratio limits may be considered as one-sided limits,
e.g., the lower limit 6 may be considered a one-sided limit,
meaning no less than 6, or 6 or greater than 6. Sub ranges
defined by a lower limit selected from the Table and an upper
limit selected from the Table, even if not on the same row of
the Table, are included.
Illustrative values of height γ, millimeters
(range limits, endpoints inclusive)

| Lower | Upper |
|---|---|
| 6 | 8 |
| 8 | 10 |
| 10 | 12 |
| 12 | 14 |
| 14 | 16 |
| 16 | 18 |
| 18 | 20 |
| 20 | 22 |
| 22 | 24 |
| 24 | 26 |
| 26 | 28 |
| 28 | 30 |
| 30 | 32 |
| 32 | 34 |
| 34 | 36 |
| 36 | 38 |
| 38 | 40 |
| 40 | 42 |
| 42 | 44 |
| 44 | 46 |
| 46 | 48 |
| 48 | 50 |
| 50 | 52 |
| 52 | 54 |
| 54 | 56 |
| 56 | 58 |
| 58 | 60 |
| 60 | 62 |
| 62 | 64 |
| 64 | 66 |
| 66 | 68 |
| 68 | 70 |
| 70 | 72 |
| 72 | 74 |
| 74 | 76 |
| 76 | 78 |
| 78 | 80 |
| 80 | 82 |
| 82 | 84 |
| 84 | 86 |
| 86 | 88 |
| 88 | 90 |
| 90 | 92 |
| 92 | 94 |
| 94 | 96 |
| 96 | 98 |
| 98 | 100 |
| 100 | 102 |
| 102 | 104 |
| 104 | 106 |
| 106 | 108 |
| 108 | 110 |
| 110 | 112 |
| 112 | 114 |
| 114 | 116 |
| 116 | 118 |
| 118 | 120 |
| 120 | — |

A hole centroid may be located at latitudinal angle Λ (lambda). Λ may correspond to a width of a perimeter region (shown in FIG. 2). Λ may have any suitable value. Table 7 shows illustrative values of Λ. Different holes may be positioned at different angles Λ.

TABLE 7
Illustrative values of Λ. Ranges defined
by any two values from the Table are included
Illustrative values of Λ, degrees of arc.

0.5
1
2
3
4
5
6
7
8
9
10
11
12
13
14
15
16
17
18
19
20
21
22
23
24
25
26
27
28
29
30
31
32
33
34
35
36
37
38
39
40
41
42
43
44
45
46
47
48
49
50
51
52
53
54
55
56
57
58
59
60
61
62
63
64
65
66
67
68
69
70
71
72
73
74
75
76
77
78
79
80
81
82
83
84
85
86
87
88
89
90

The prosthesis may be provided in a kit that includes a plurality of prosthesis having different dimensions.

FIG. 4 shows a bottom view of prosthesis 200.

FIG. 5 shows schematically illustrative prosthesis 500 in an oblique view that is analogous to that shown in FIG. 4, of prosthesis 200. Apex a′ of seating surface 502 is displaced from apex a″ of the replacement articular surface (in background). Edge 504 of the replacement articular surface is not geometrically similar to edge 506 of seating surface 502.

FIG. 6 shows schematically illustrative prosthesis 600 in a view that is analogous to that shown in FIG. 5, of prosthesis 500. Edge 604 of the replacement articular surface is oval. Edge 606 of seating surface 602 is irregular.

FIG. 7 shows schematically illustrative prosthesis 700 in a view that is analogous to that shown in FIG. 3, of prosthesis 200.

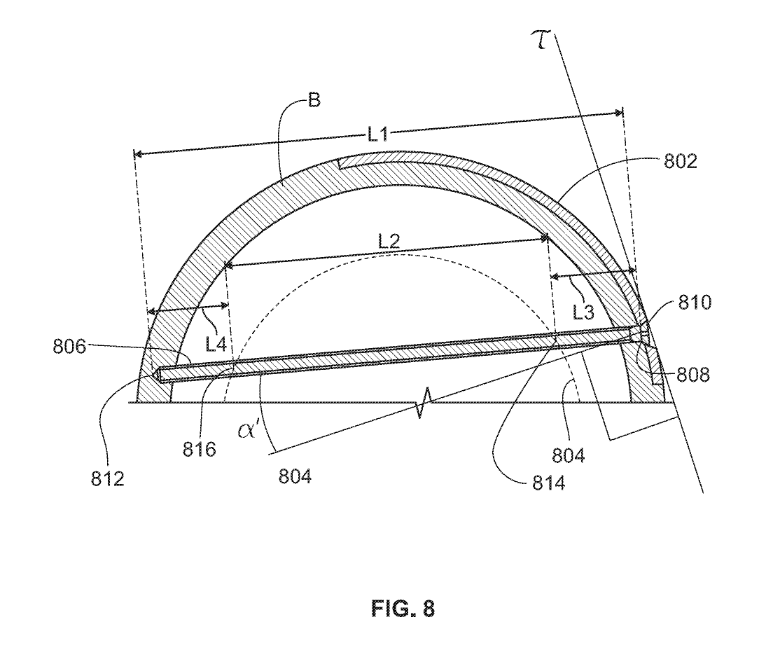

FIG. 8 shows schematically a cross-section of illustrative arrangement 800. Arrangement 800 may include prosthesis 802, anchoring hull 804, and one or more screws 806. In some orthopedic fixation techniques, a practitioner uses fluoroscopic or mechanical registration (e.g., jigs) to establish a screw trajectory between an entry point on exposed tissue and an individual predetermined reception point in an implanted structure. Because the anchoring hull can receive the screw at one of many different openings that are distributed over the surface of the hull, and because the openings have tolerance to receive the screw along trajectories having different angles relative to the hull surface, such registration may be avoided.

Length L₁ is defined as running from base 808 of screw head 810 to screw tip 812. Length L₂ is defined as running between point 814 on screw 806, at which screw 806 intersects with an entry point on anchoring hull 804 and point 816 on screw 806, at which screw 806 intersects with an exit point on anchoring hull 804. Length L₃ is defined as running between base 808 and point 814. Length L₄ is defined as running between point 816 and screw tip 812.

Lengths L₁, L₂, L₃ and L₄ will depend on, among other things, the shape and size of bone B, the size of anchoring hull 804, the position of anchoring hull 804 in bone B, the shape of anchoring hull 804, and the angle α′ (shown also in FIG. 2), between screw-hole tangent τ and screw-hole central axis z.

Relative values of lengths L₁, L₂, L₃ and L₄ may distribute torque on the screw, and thus avoid or reduce torque and bending load concentrations that may lead to failure.

Illustrative ratios of lengths L₁, L₂, L₃ and L₄ are included in Table 2.

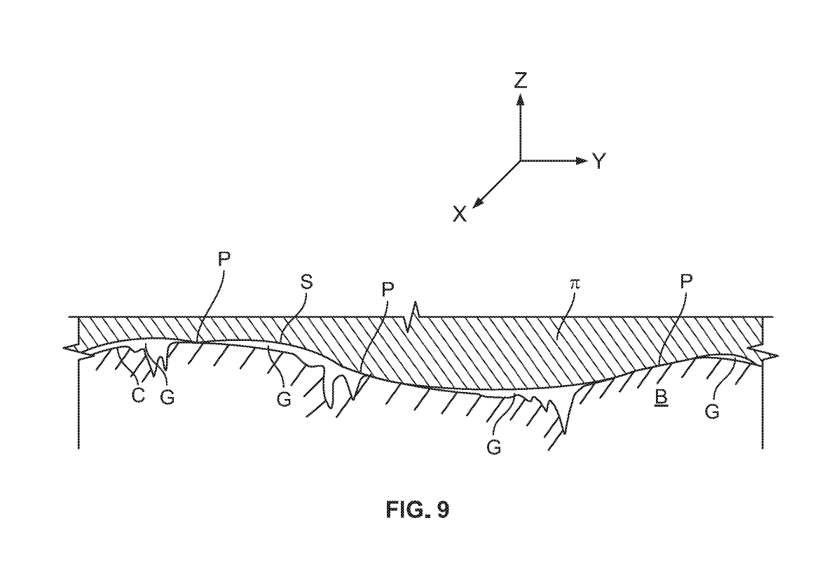

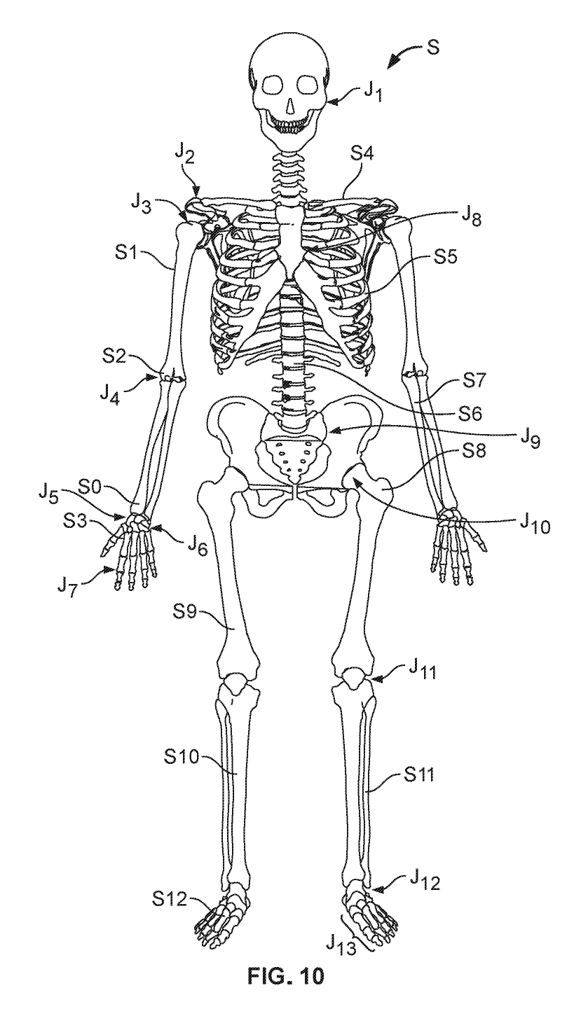

FIG. 9 shows conceptually a prosthesis Π complementarily seated against contour C of a prepared surface on bone B. The prepared surface may intersect fractures in bone B. This may leave gaps or voids in the prepared surface. Contour C may have a height z that varies in one or both of directions x and y. Prosthesis Π has seating surface S that complements contour C.

Gaps G may intervene between seating surface S and contour C. S may be complementarily seated against C based on discrete points of contact P. The discrete points of contact may be spaced apart from each other.

Seating surface S may be coincident with contour C. When seating surface S is coincident with contour C, no gaps may intervene between seating surface S and contour C.

Bone B may be monolithic. Bone B may be fractured. The prepared surface may have properties that depend on an instrument or approach used to shape the prepared surface. The approach may include reaming. The approach may include use of an osteotome.

FIG. 10 shows illustrative anatomy in connection with which the apparatus and methods may be used. FIG. 3 shows illustrative skeleton S. Skeleton S may include illustrative bones Sᵢ in which apparatus and methods in accordance with the principles of the invention may be used. The apparatus and methods may be used in connection with “hollow” bones. The hollow bones may include cortical tissue. The hollow bones may include cancellous tissue. Cortical tissue may be referred to as “tissue.” Cancellous tissue may be referred to as “tissue.” Other matter in the interior of a bone may be considered “tissue.” The bone may be considered “tissue.”

The apparatus and methods may be used to create a space inside a bone. The space may be a cavity. The tissue may be inside the bone. The space may be created by breaking up the tissue. The space may be created by removing the tissue from the bone. The space may be created as part of a therapeutic procedure. The apparatus and methods may displace tissue by imparting mechanical energy to the tissue, for example, through one or more of expanding motion, rotational motion, axial motion, compressive motion, cutting motion, and any other suitable motions.

The apparatus and methods may be used to deploy an anchoring hull in the space created inside the bone. The apparatus and methods may be used to anchor the anchoring hull to the bone.

Illustrative bones Sᵢ in which apparatus and methods in accordance with the principles of the invention may be used are included in Table 9 below.

TABLE 9
Bones Sᵢ.

| | Reference numeral |
|---|---|
| Bone | in FIG. 3 |
| Distal Radius | S₀ |
| Humerus | S₁ |
| Proximal Radius and Ulna (Elbow) | S₂ |
| Metacarpals | S₃ |
| Clavicle | S₄ |
| Ribs | S₅ |
| Vertebrae | S₆ |
| Ulna | S₇ |
| Hip | S₈ |
| Femur | S₉ |
| Tibia | S₁₀ |
| Fibula | S₁₁ |
| Metatarsals | S₁₂ |

Illustrative joints Jᵢ in which apparatus and methods in accordance with principles of the invention may be used are included in Table 10 below. Table 10 may include a partial list of joints Jᵢ.

TABLE 10
Illustrative joints Ji.

| | Reference numeral |
|---|---|
| Joint | in FIG. 4 |
| Temporomandibular Joint | J₁ |
| Acromioclavicular Joint | J₂ |
| Shoulder | J₃ |
| Elbow | J₄ |
| Wrist | J₅ |
| Carpometacarpal Joint | J₆ |
| Metacarpophalangeal Joint | J₇ |
| Sternoclavicular Joint | J₈ |
| Sacroiliac Joint | J₉ |
| Hip Joint | J₁₀ |
| Knee Joint | J₁₁ |
| Ankle | J₁₂ |
| Interphalangeal articulations of foot | J₁₃ |

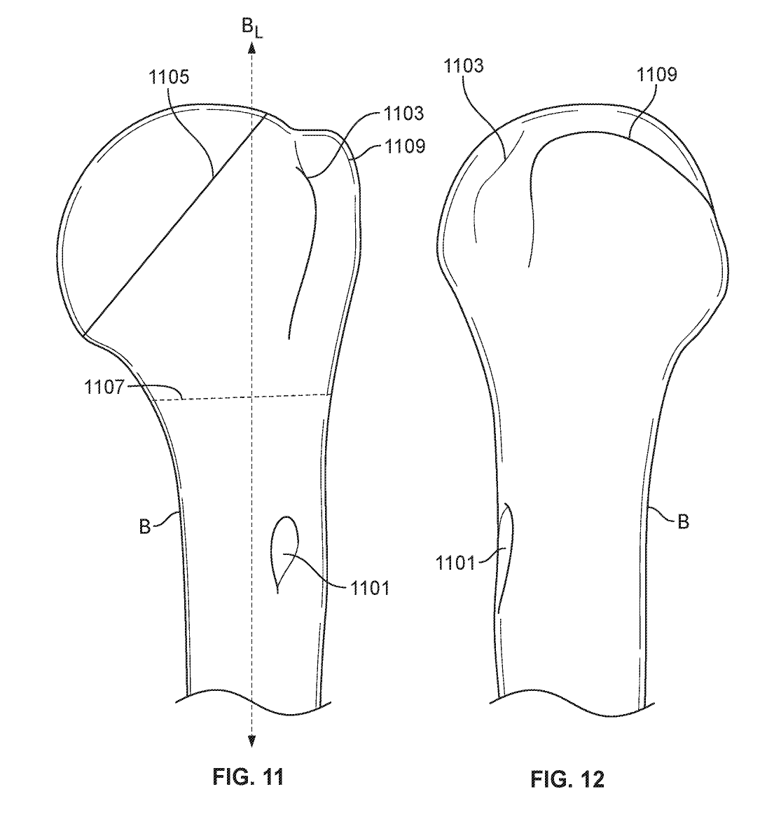

FIG. 11 shows a lateral view of bone B. In FIG. 11, bone B is a humerus. A proximal portion of the humerus is illustrated in FIG. 11. For the purposes of the application, “proximal” may refer to a location closer to a patient's core, and “distal” may refer to a location further away from the patient's core. A proximal portion of the humerus may be referred to herein as the proximal humerus.

The proximal humerus may include greater tuberosity 1109, lesser tuberosity 1103, surgical neck 1107, head of humerus 1105 and deltoid tuberosity 1101, among other features. B_{L} is a longitudinal axis defined by bone B.

FIG. 12 shows a view of the proximal humerus that is different from the view illustrated in FIG. 2A.

FIGS. 13-19 show illustrative apparatus and methods for implanting illustrative anchoring hull 1301 in a bone B and seating illustrative prosthesis 1501 on a prepared surface RS of bone B. Anchoring hull 1301 may include one or more features of anchoring hulls described herein. Prosthesis 1501 may include one or more features of prostheses described herein. The methods illustrated in FIGS. 13-19 are not limiting, and may include other method steps described herein.

FIG. 13 shows illustrative anchoring hull 1301 implanted antegrade in an interior of bone B. Bone B may define articular surface AS.

In FIG. 13, screws 1307 and 1309 are shown anchoring tail 1305 of anchoring hull 1301 to a shaft of bone B.

In FIG. 13, screw 1311 is shown passing through bone B and into a head of anchoring hull 1301. Screw 1313 is shown passing through a tuberosity in bone B and into a head of anchoring hull 1301. One or both of screws 1311 and 1313 may be used by a practitioner to reduce a fracture in bone B (fracture not shown).

Articular surface AS may define apex AS_{A}. An axis passing through apex AS_{A} and transecting longitudinal axis Z may be offset from longitudinal axis Z by angle θ_{AS}.

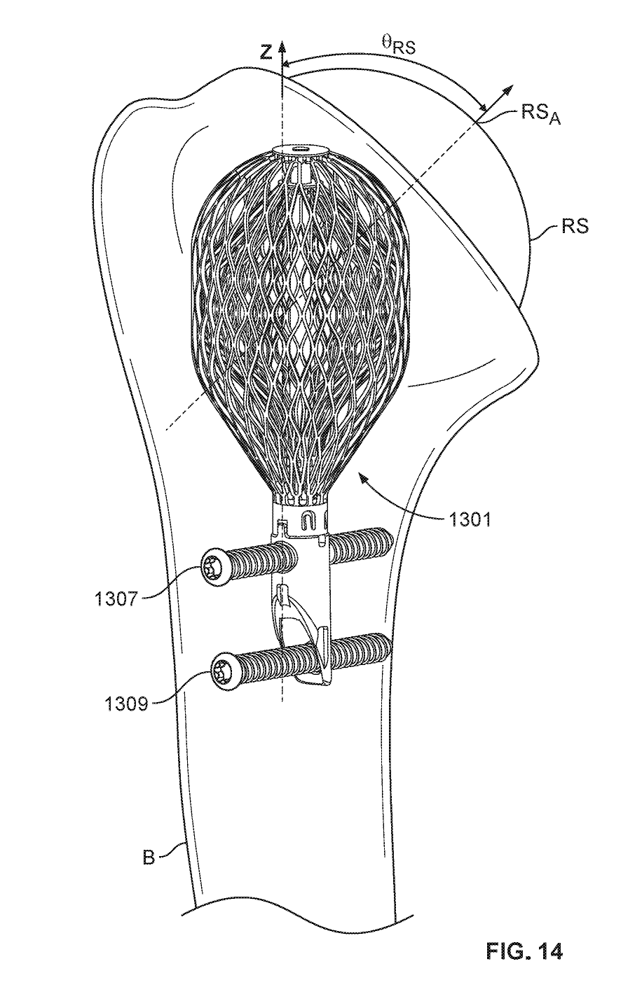

FIG. 14 show anchoring hull 1301 implanted in bone B with screws 107 and 109 anchoring tail 1305 of anchoring hull 1301 to a shaft of bone B.

In FIG. 14, native articular bone on articular surface AS of bone B (shown in FIG. 13) has been removed to form prepared surface RS. Prepared surface RS may be prepared to receive a prosthesis.

During a procedure in which the articular surface of bone B has been fractured, preparation of prepared surface RS may include reducing segments of the articular surface.

Prepared surface RS may define an apex RS_{A}. An axis passing through apex RS_{A} and transecting longitudinal axis Z may be offset from longitudinal axis Z by angle θ_{RS}.

Angle θ_{AS} may be offset from angle θ_{RS} by a value ranging between 0° and 15°. A practitioner may select an area on articular surface AS for preparing the reduced surface RS, the selected area having an apex RS_{A} that is offset from the articular surface apex AS_{A} from a continuous range of 0° and 15°. Thus, the practitioner is not limited to predetermined areas on the articular surface for preparing the reduced articular surface, and instead can select from a plurality of continuously distributed areas on articular surface AS.

In FIG. 14, illustrative prepared surface RS is smooth. However, prepared surface RS may be jagged, bumpy, rocky, or include one or more depressions. Prepared surface RS may be prepared using apparatus mentioned herein. A contour of prepared surface RS may depend at least in part on apparatus used to prepare prepared surface RS.

The implanting of anchoring hull 1301 in bone B may be performed during a clinical procedure that includes the preparation of prepared surface RS.

The implanting of anchoring hull 1301 into bone B may be performed during a first surgical procedure. Preparation of prepared articular surface AS may be performed during a second surgical procedure after completion of the first surgical procedure.

FIGS. 14-19 only illustrate screws that pass through prepared surface RS, but screws (not shown) may be driven through bone outside of RS, such as the tuberosities, and into anchoring hull 1301. The methods illustrated in one or more of FIGS. 14, 15, 16, 17, 18, and 19 may be performed when one or more screws are anchored both on a surface of bone B not including prepared surface RS and in anchoring hull 1301.

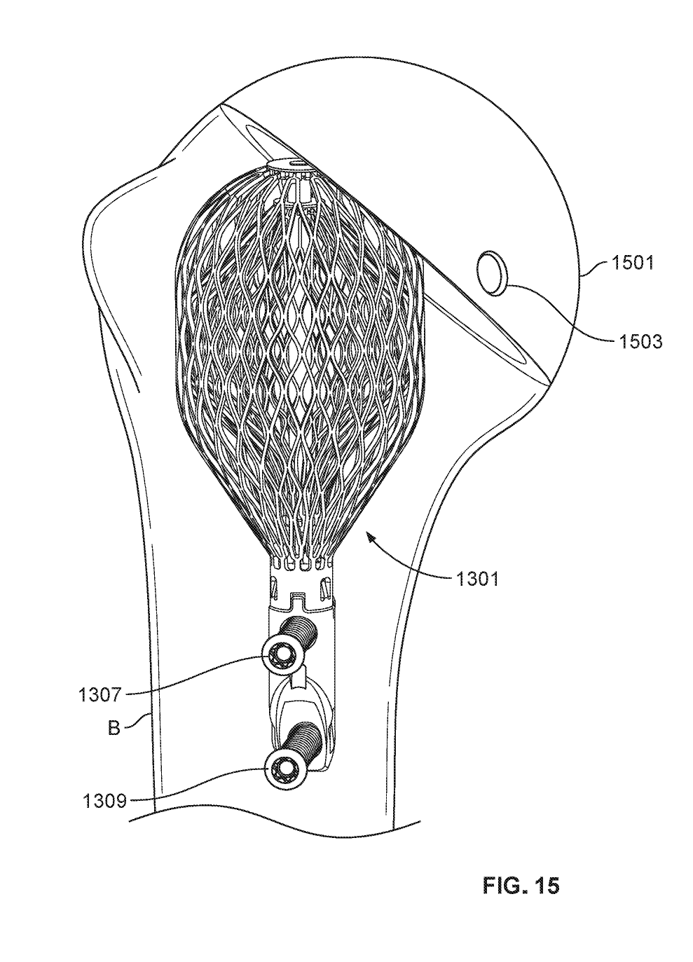

FIG. 15 shows illustrative prosthesis 1501 seated on the prepared surface RS (illustrated in FIG. 14). Prosthesis 1501 may be seated on the prepared surface RS such that a layer of native bone separates the entire prosthesis 1501 from anchoring hull 1301.

Anchoring hull 1301 may be implanted into bone B during a surgical procedure that includes placing prosthesis 1501 on the prepared surface.

The implanting of anchoring hull 1301 into bone B, and the fixing of the tuberosity to anchoring hull 1301 with screws 1313 and 1315, may be performed during a first surgical procedure. Prosthesis 1501 may be seated on bone B during a second surgical procedure performed after completion of the first surgical procedure.

Prosthesis 1501 may include hole 1503. Prosthesis 1501 may include one or two additional holes (not shown). Holes in prosthesis 1501 may be positioned adjacent an edge of prosthesis 1501.

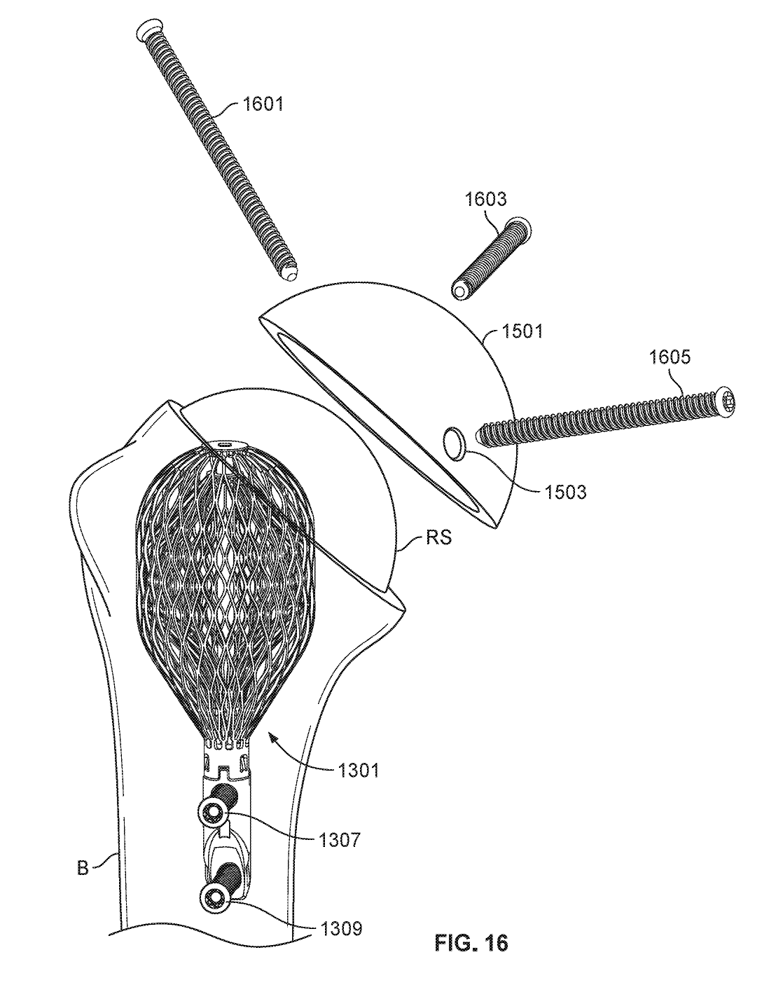

FIG. 16 shows illustrative prosthesis 1501 prior to placement on prepared surface RS. Prosthesis 1501 may be placed on prepared surface RS using a jig (not shown). Prosthesis 1501 may be placed on prepared surface RS without using a jig.

Prosthesis 1501 may include hole 1503 and two additional holes (not shown). Screw 1605 may be inserted into hole 1503. Screws 1601 and 1603 may be inserted into the two additional holes.

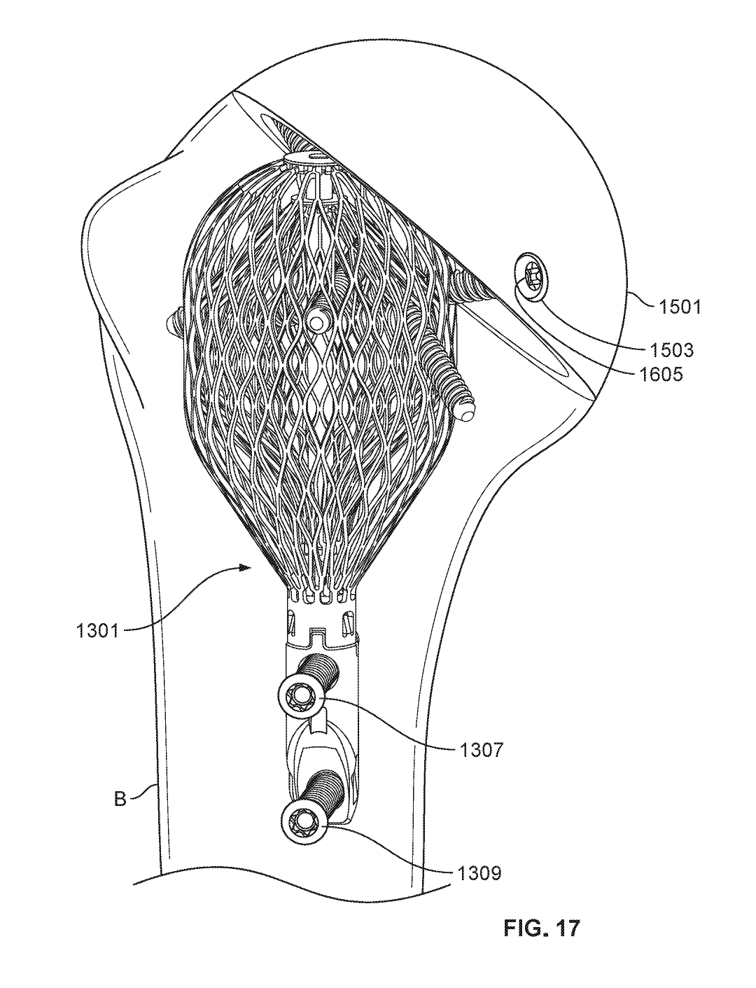

FIG. 17 shows prosthesis 1501 positioned on prepared surface RS and anchored to anchoring hull 1301. Screws 1601, 1603 and 1605 are illustrated as extending through holes in the prosthesis 1501, through first openings in anchoring hull 1301, through a volume defined by anchoring hull 1301 and through second openings in anchoring hull 1301.

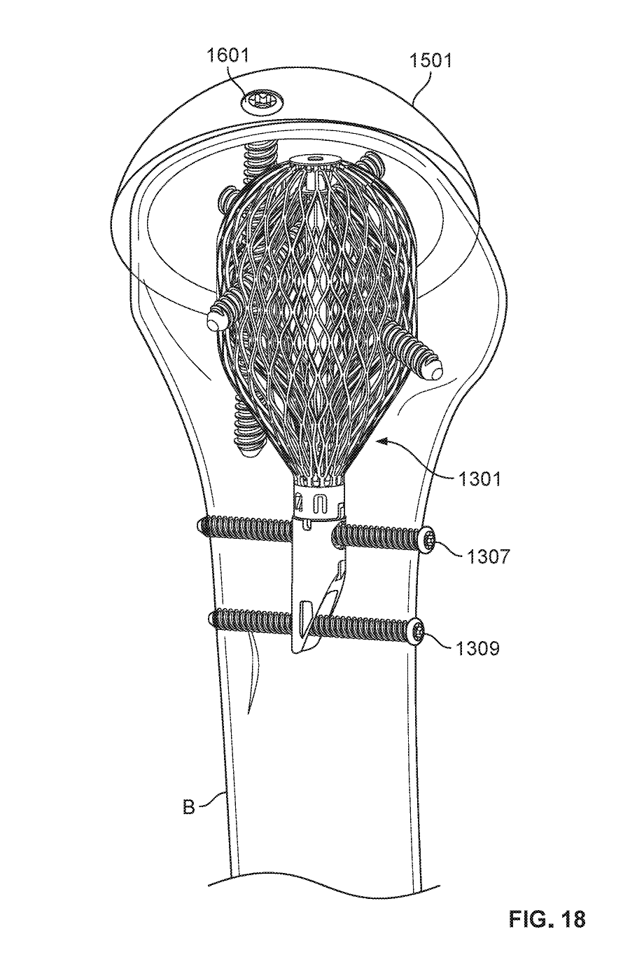

FIG. 18 shows another view of prosthesis 1501 positioned on prepared surface RS and anchored to anchoring hull 1301 with screws 1601, 1603 and 1605 extending through holes in prosthesis 1501 and into anchoring hull 1301.

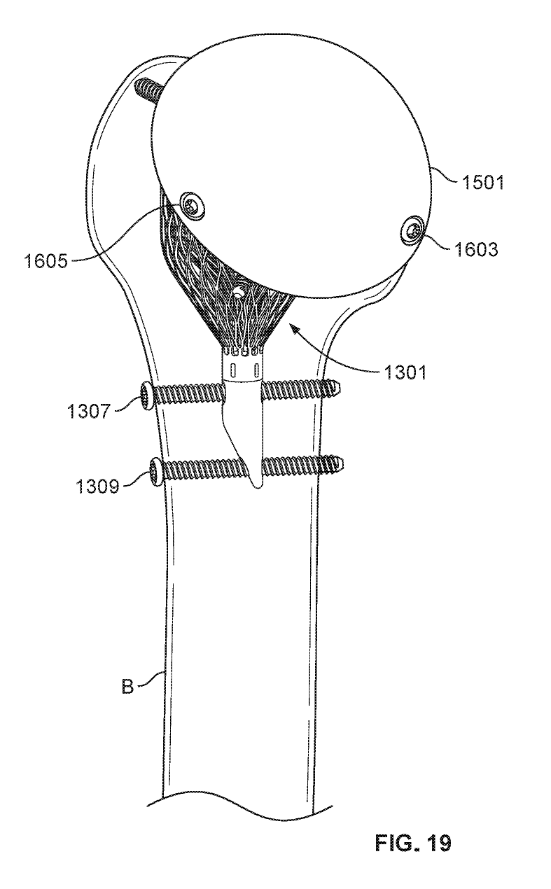

FIG. 19 shows another view of prosthesis 1501 positioned on prepared surface RS and anchored to anchoring hull 1301 with screws 1601, 1603 and 1605 extending through holes in prosthesis 1501 and into anchoring hull 1301.

FIGS. 20-26 show illustrative apparatus and methods for implanting illustrative anchoring hull 2001 in a bone B and seating illustrative prosthesis 2201 on a prepared surface RS of bone B. Anchoring hull 2001 may include one or more features of anchoring hulls described herein. Prosthesis 2201 may include one or more features of prostheses described herein. The methods illustrated in FIGS. 20-26 are not limiting, and may include other method steps described herein.

FIG. 20 shows illustrative anchoring hull 2001 implanted retrograde in bone B. Bone B may include articular surface AS. Anchoring hull 2001 may include head 2003 and tail 2005. Illustrative screws 2007 and 2009 are shown engaged with tail 2005 of anchoring hull to anchor anchoring hull 2001 to bone B.

FIGS. 20-26 only illustrate screws that pass through prepared surface RS, but screws (not shown) may be driven through bone outside of RS, such as the tuberosities, and into anchoring hull 2001. The methods illustrated in one or more of FIGS. 20, 21, 22, 23, 24, 25 and 26 may be performed when one or more screws are anchored both on a surface of bone B not including prepared surface RS and in anchoring hull 2001.

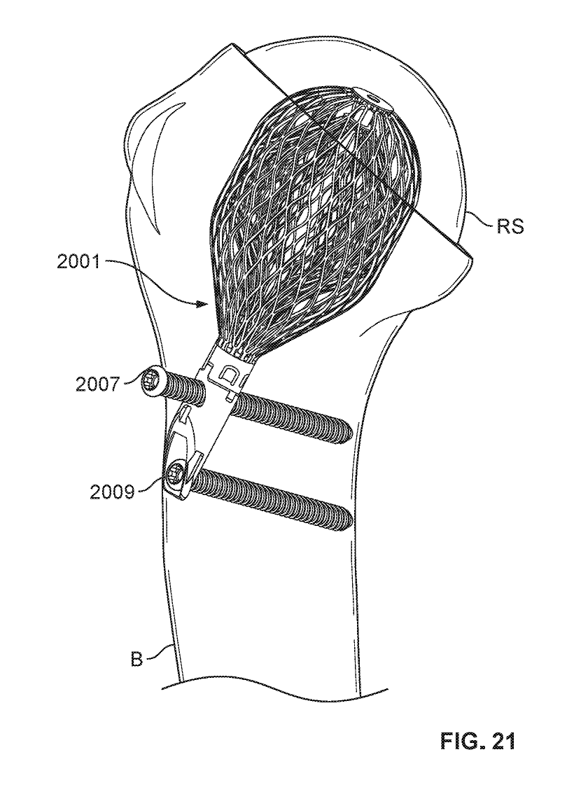

FIG. 21 shows native articular bone on articular surface AS of bone B (shown in FIG. 20) removed to form prepared surface RS. Prepared surface RS may be prepared to receive a prosthesis.

In the event that articular surface AS is fractured, preparation of prepared surface RS may include reducing segments of articular surface AS.

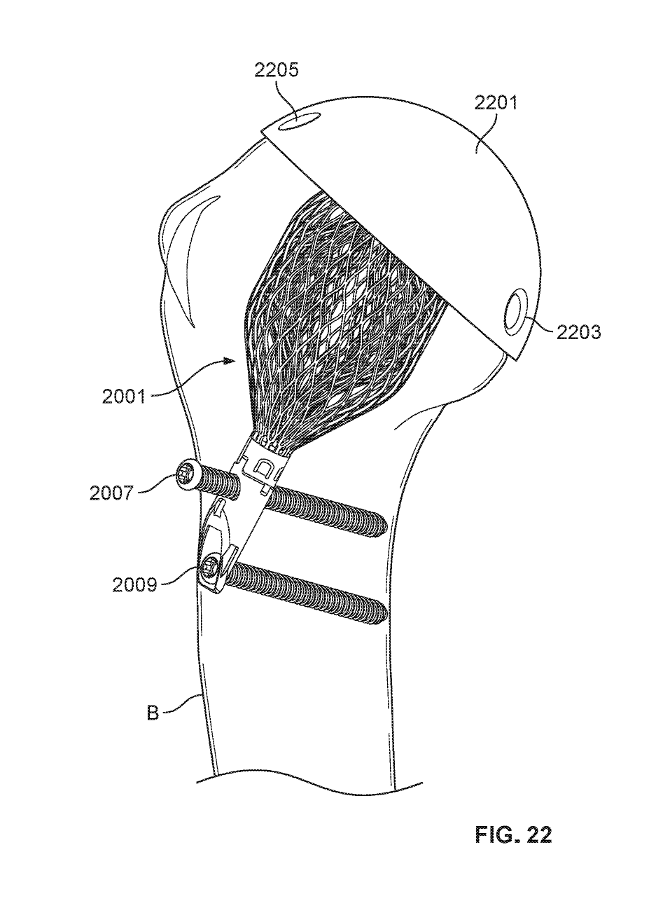

FIG. 22 shows illustrative prosthesis 2201 seated on prepared surface RS (illustrated in FIG. 21). Prosthesis 2201 may be seated on prepared articular surface AS such that a layer of native bone separates the entire prosthesis 2201 from anchoring hull 2001.

Anchoring hull 2001 may be implanted in bone B, and prosthesis 2201 may be seated on bone B, during the same surgical procedure.

The implanting of anchoring hull 2001 into bone B may be performed during a first surgical procedure. Prosthesis 2201 may be seated on bone B during a second surgical procedure performed after completion of the first surgical procedure.

Prosthesis 2201 may include hole 2203 and hole 2205. Prosthesis 1501 may include an additional hole (not shown). Holes in prosthesis 2201 may be positioned adjacent an edge of prosthesis 1501.

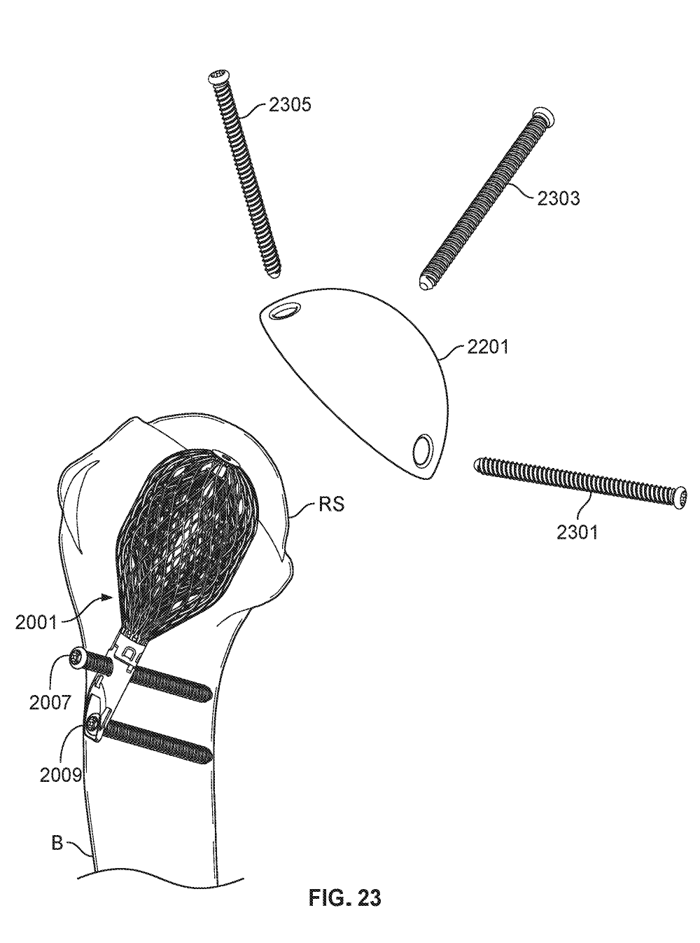

FIG. 23 shows illustrative prosthesis 2201 prior to placement on prepared surface RS.

Prosthesis 2201 may be placed on prepared surface RS using a jig (not shown).

Prosthesis 2201 may be placed on prepared surface RS without using a jig.

Prosthesis 2201 may have three holes. Screws 2301, 2303 and 2305 may be inserted into the holes.

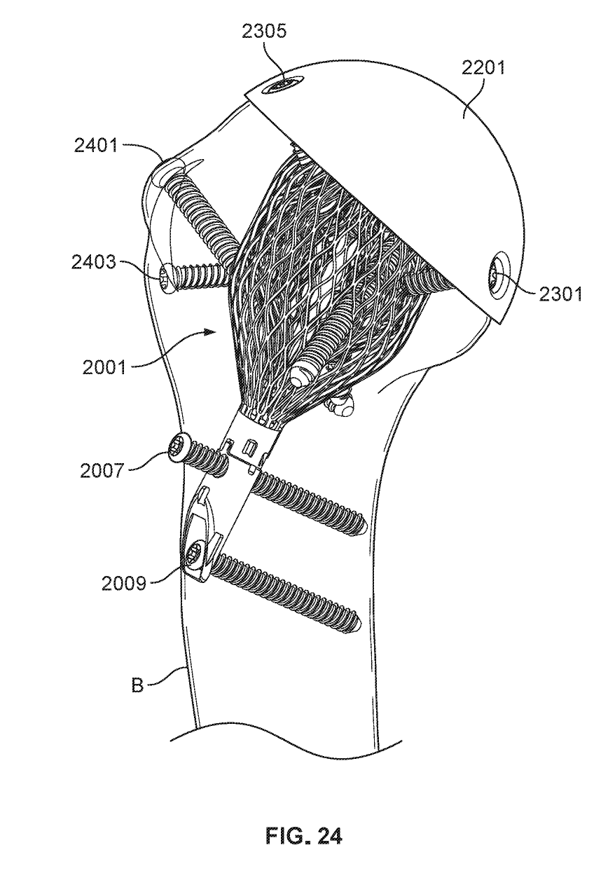

FIG. 24 shows prosthesis 2201 positioned on prepared surface RS and anchored to anchoring hull 2001. Screws 2301, 2303 and 2305 are illustrated as extending through holes in prosthesis 2201, through first openings in anchoring hull 2001, through a volume defined by anchoring hull 2001 and through second openings in anchoring hull 2001.

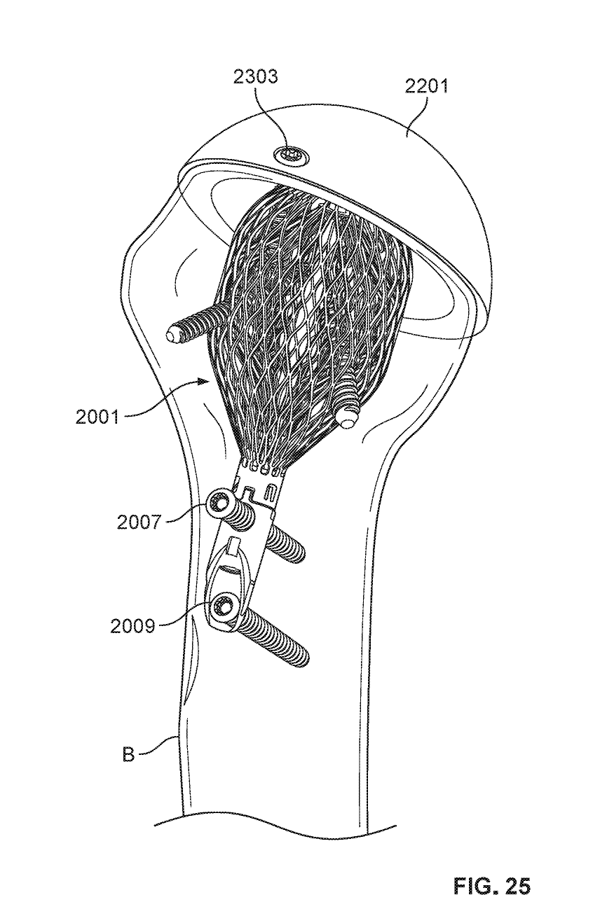

FIG. 25 shows another view of prosthesis 2201 positioned on prepared surface RS and anchored to anchoring hull 2001 with screws 2301, 2303 and 2305 extending through holes in the prosthesis 2201 and into anchoring hull 2001.

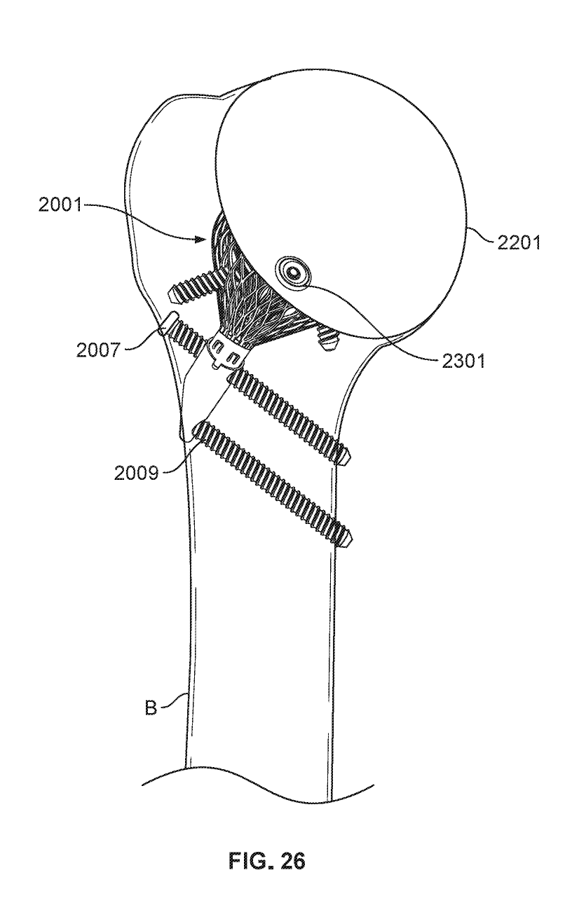

FIG. 26 shows another view of prosthesis 2201 positioned on prepared surface RS and anchored to anchoring hull 2001 with screws 2301, 2303 and 2305 extending through holes in the prosthesis 2201 and into anchoring hull 2001.

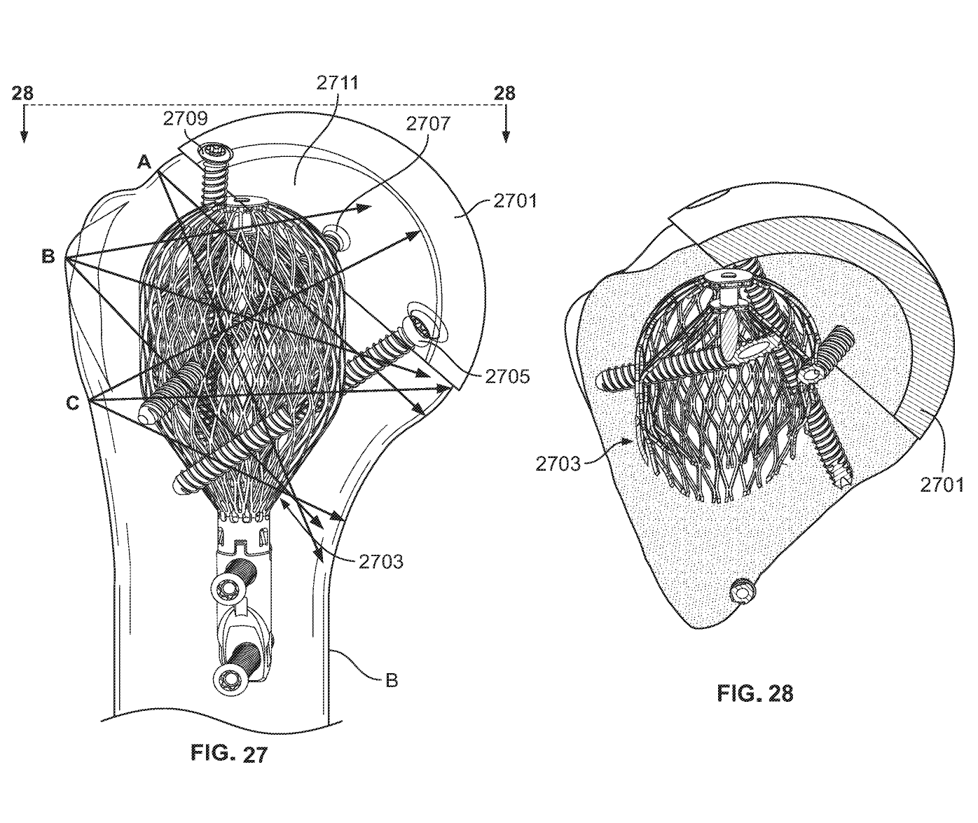

FIG. 27 shows illustrative anchoring hull 2703 implanted retrograde in bone B. FIG. 27 also shows illustrative prosthesis 2701 positioned on a prepared surface of bone B. The prepared surface may be formed by removing native articular surface from an articular surface of bone B.

Screws 2709, 2707 and 2705 are shown seated in holes in prosthesis 2701 and engaging openings in anchoring hull 2703.

Prosthesis 2701 is illustrated as transparent in FIG. 27 to show a layer of bone 2711 separating the entire prosthesis 2701 from anchoring hull 2703.

Illustrative positions along bone B for driving screws through bone B and into anchoring hull 2701 are shown at points A, B and C. Illustrative angles for driving screws through points A, B and C are shown using arrows extending away from each of points A, B and C and into bone B.

FIG. 28 shows a cross-sectional view of apparatus illustrated in FIG. 27 taken along lines 28-28.

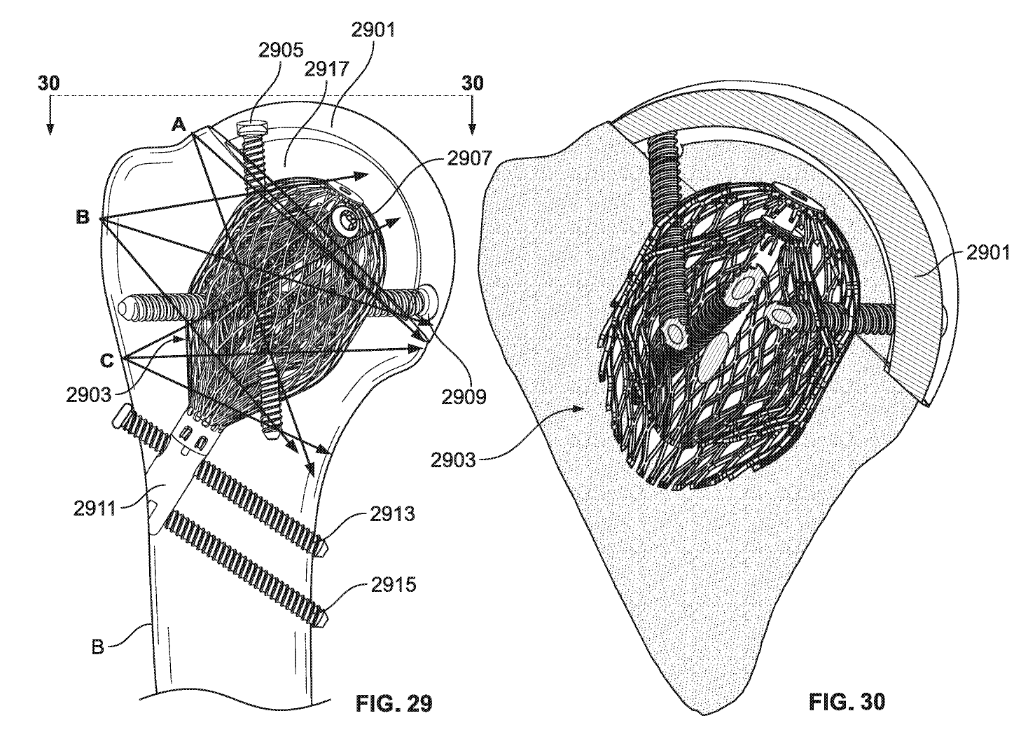

FIG. 29 shows illustrative anchoring hull 2903 implanted antegrade in bone B. FIG. 29 also shows illustrative prosthesis 2901 positioned on a prepared surface of bone B (not shown). The prepared surface may be formed by removing native articular surface from an articular surface of bone B.

Screws 2909, 2907 and 2905 are shown seated in holes in prosthesis 2701 and engaging openings in anchoring hull 2703. Screws 2913 and 2915 are shown engaging tail 2911 of anchoring hull 2903.

Prosthesis 2901 is illustrated as transparent in FIG. 29 to show a layer of bone 2917 separating the entire prosthesis 2901 from anchoring hull 2903.

Illustrative positions along bone B for driving screws through bone B and into anchoring hull 2901 are shown at points A, B and C. Illustrative angles for driving screws through points A, B and C are shown using arrows extending away from each of points A, B and C and into bone B.

FIG. 30 shows a cross-sectional view of apparatus illustrated in FIG. 29 taken along lines 30-30.

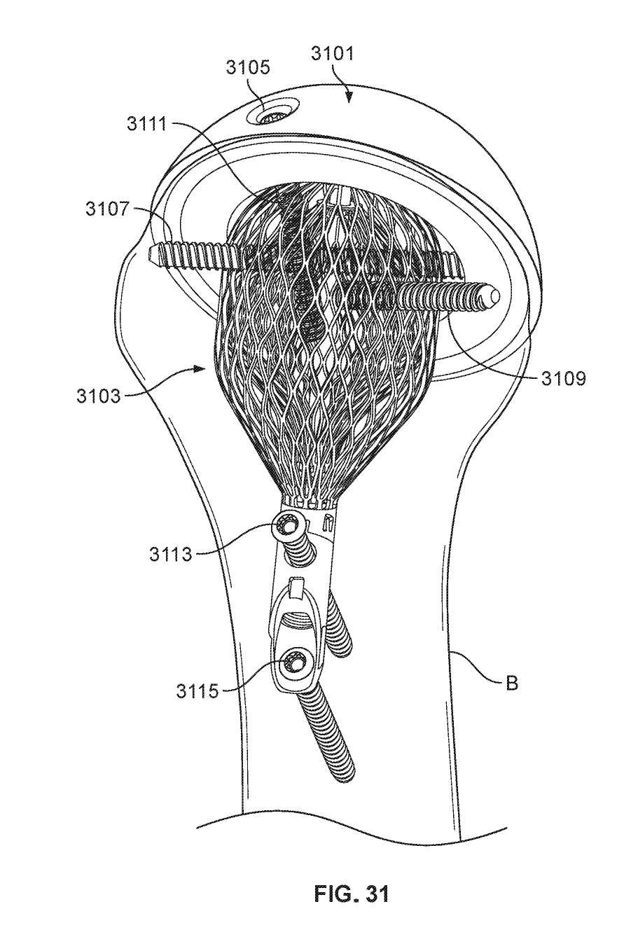

FIG. 31 shows illustrative anchoring hull 3103 implanted retrograde in bone B. FIG. 31 also shows illustrative prosthesis 3101 positioned on a prepared surface of bone B (not shown). The prepared surface may be formed by removing native articular surface from an articular surface of bone B.

Prosthesis has hole 3105 and two additional holes (not shown). Screw 3111 is shown seated in hole 3105. Screws 3107 and 3109 are seated in the two additional holes.

Screws 3113 and 3115 are shown engaging a tail of anchoring hull 3103.

Illustrative prosthesis 3101 abuts anchoring hull 3103. Abutment of prosthesis 3101 to anchoring hull 3103 may not anchor prosthesis 3101 to anchoring hull 3103 at the place of the abutment. Screws 3111, 3107 and 3109 are therapeutic devices used to anchor prosthesis 3101 to anchoring hull 3103.

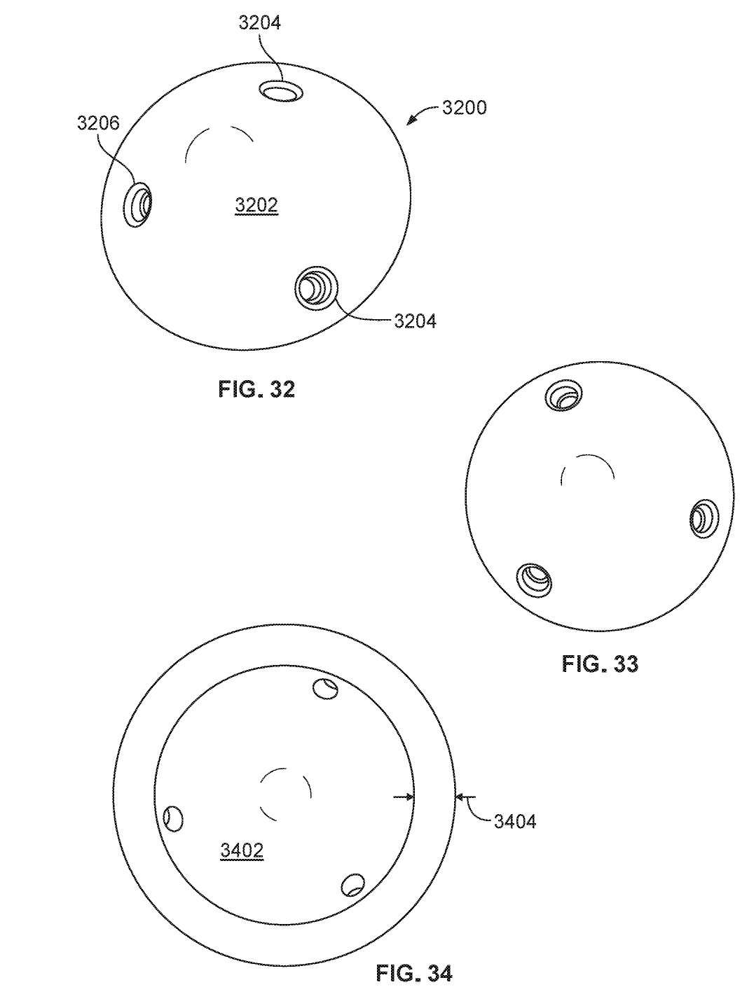

FIG. 32 shows illustrative prosthesis 3200 in perspective. Prosthesis 3200 may be dome-shaped. Prosthesis 3200 may include replacement articular surface 3202. Prosthesis 3200 may include holes 3204 and hole 3206.

FIG. 33 shows prosthesis 3200 in top view.

FIG. 34 shows prosthesis 3200 in bottom view. Prosthesis 3200 may have seating surface 3402. Surface 3402 may be dome-shaped. Prosthesis 3200 may have thickness 3404.

Thus, apparatus and methods for articular surface repair have been provided. Persons skilled in the art will appreciate that the present invention can be practiced by other than the described examples, which are presented for purposes of illustration rather than of limitation. The present invention is limited only by the claims that follow.

1. Apparatus for repairing a bone, the apparatus comprising a prosthesis:
having:
a first face shaped to conform to a native articular bone surface and defining a perimeter; and
a second face shaped to conform to a bone surface prepared for receiving the prosthesis; and
defining two or more screw-holes adjacent the perimeter.

2. The apparatus of claim 1 wherein the prosthesis defines, other than the screw-holes, no anchor pass-through.

3. The apparatus of claim 1 wherein:
the prosthesis comprises a thickness between the first face and the second face;
a normal to the first face points in a first direction;
a normal to the second face points in a second direction; and
the first and second directions diverge.

4. The apparatus of claim 1 wherein the prosthesis does not include an anchoring stem.

5. The apparatus of claim 1 wherein the prosthesis does not include a fixture for receiving an anchoring stem.

6-7. (canceled)

8. The apparatus of claim 1 wherein the two or more holes are sized to receive a screw.

9-10. (canceled)

11. The apparatus of claim 1 wherein each of the two or more holes define a first opening on the first face and a second opening on the second face, the first opening being larger than the second opening.

12. The apparatus of claim 1 wherein the second face is smooth.

13. The apparatus of claim 1 wherein the prosthesis is dome-shaped.

14. The apparatus of claim 13 wherein the prosthesis defines a truncated sphere.

15. The apparatus of claim 1 wherein the first surface has a topography that includes one or more valleys and one or more hills.

16. The apparatus of claim 1 wherein:
the two or more holes include:
a first hole having a diameter and a centroid that is at a first distance from the perimeter;
a second hole having a diameter and a centroid that is at a second distance from the perimeter;
a third hole having a diameter and a centroid that is at a third distance from the perimeter; and
each of the first, second and third distances is no less than 1.5 times the diameter.

17-33. (canceled)

34. The apparatus of claim 1 wherein the two or more holes are positioned, along a path that contours the perimeter, at uniform offsets from each other.

35. The apparatus of claim 1 further comprising:
an anchoring hull defining a plurality of openings;
wherein:
the prosthesis and the anchoring hull are configured to be fixed together by screws or one or more tension-applying members; and
direct physical contact between the prosthesis and the anchoring hull, therapeutically in situ, does not effect an anchoring of the prosthesis to the anchoring hull.

36. The apparatus of claim 35 wherein the prosthesis is not configured to mate with the anchoring hull.

37. The apparatus of claim 35 wherein the prosthesis is not configured to snap into the anchoring hull.

38. The apparatus of claim 35 wherein an abutment of the prosthesis against the anchoring hull does not rigidly couple the prosthesis to the anchoring hull.

39. The apparatus of claim 35 wherein the plurality of openings are sized to receive screws having a length in the range 10 mm to 120 mm and a diameter in the range 1.1 mm to 10 mm.

40. The apparatus of claim 35 wherein:
the plurality of openings defined by the anchoring hull has a first number of openings;
the two or more holes defined by the prosthesis have a second number of openings; and
the first number is at least ten times the second number.

41. Apparatus for repairing a bone, the apparatus comprising:
first means for performing a function of native bone tissue;
second means for anchoring the first means to the bone; and
third means for fastening the first means to the second means.

42. (canceled)

43. A method for repairing a bone, the method comprising:
implanting an anchoring hull in an interior of the bone;
removing native articular bone to provide a prepared surface upon which to seat a prosthesis; and
seating the prosthesis on the prepared surface in a position in which the prosthesis is entirely spaced apart from the anchoring hull by a layer of native bone.

44-109. (canceled)

110. A method for repairing a bone the method comprising:
implanting, in an interior of the bone, an anchoring hull;
preparing an articular surface of the bone for receiving a prosthesis; and
placing a prosthesis on the prepared surface;
wherein:
the prosthesis abuts the anchoring hull; and
the abutment does not provide engagement between the prosthesis and the hull.

111-114. (canceled)
